(19) United States
(12) Patent Application Publication
(10) Pub. No.: US 2025/0127929 A1
(43) Pub. Date: Apr. 24, 2025

(54) SCAFFOLDS FOR MODIFYING IMMUNE CELLS AND THE USES THEREOF

(71) Applicants: President and Fellows of Harvard College, Cambridge, MA (US); The General Hospital Corporation, Boston, MA (US)

(72) Inventors: David T. Scadden, Weston, MA (US); David J. Mooney, Cambridge, MA (US)

(73) Assignees: President and Fellows of Harvard College, Cambridge, MA (US); The General Hospital Corporation, Boston, MA (US)

(21) Appl. No.: 18/899,943

(22) Filed: Sep. 27, 2024

Related U.S. Application Data
(63) Continuation of application No. PCT/US2023/016724, filed on Mar. 29, 2023.
(60) Provisional application No. 63/325,100, filed on Mar. 29, 2022.

(51) Int. Cl. A61K 48/00; C12N 9/22; C12N 15/11; C12N 15/90

(52) U.S. Cl. CPC A61K 48/0058; A61K 48/0025; A61K 48/0075; C12N 9/22; C12N 15/11; C12N 15/907; C12N 2310/20

(57) ABSTRACT

The present invention provides compositions and methods for modifying immune cells in a subject.

### RELATED APPLICATIONS

The instant application is a continuation of PCT Application No. PCT/US2023/016724, filed on Mar. 29, 2023, which claims priority to U.S. Provisional Application No. 63/325,100, filed on Mar. 29, 2022, the entire contents of each of which are incorporated herein by reference in their entirety.

### BACKGROUND

The use of allogeneic hematopoietic stem cell transplantation (HSCT) to cure multiple disorders is limited by deficiency and dysregulation of T-cells. In allogeneic HSCT, hematopoietic stem cells (HSCs) and/or hematopoietic progenitor (HPCs) cells are harvested from the body, genetically engineered ex vivo, and re-infused into the same individual after administration of a conditioning treatment that favor their engraftment in the bone marrow. The engrafted HSCs and/or HPCs can ensure a steady supply of genetically engineered progeny potentially for the recipient's lifetime. Mature cells of different lineages may then reverse pathological conditions such as hematological disorders and cancer.

However, allogeneic HSCT can be associated with a marked deficiency in T-cell generation, which renders patients susceptible to infectious agents and may contribute to graft-versus-host disease (GVHD). These complications can be fatal and limit the use of HSCT in settings where it can be curative. Reconstitution of the naïve helper and effector T-cell subsets, along with the restoration of the T-cell receptor repertoire remains a significant unmet clinical need. See, e.g., Shah et al. Nat Biotechnol. 37:293-302, 2019.

### SUMMARY OF INVENTION

Disclosed herein are novel compositions and methods for modulating the immune system of a subject. Such compositions may be administered to a subject to inter alia locally transduce a cell, such as an immune cell (e.g., a hematopoietic stem cell (HSC) and/or a hematopoietic progenitor cell (HPC)) in a subject.

Accordingly, in one aspect, the present invention provides a method of delivering an active agent to a subject in need thereof (e.g., to a cell, such as an immune cell, in a subject in need thereof), comprising: (a) administering to a subject a scaffold composition, wherein the scaffold composition: (i) is a porous scaffold material; and (b) administering an active agent into the scaffold composition in situ, thereby delivering the active agent to the subject.

In another aspect, the present invention provides a method of locally transducing an immune cell (e.g., a hematopoietic stem cell (HSC) and/or a hematopoietic progenitor cell (HPC)) in a subject in need thereof, comprising: (a) administering to a subject a scaffold composition, thereby recruiting immune cells (e.g., hematopoietic stem cells (HSCs) and/or hematopoietic progenitor cells (HPCs)) to form a bone marrow niche, wherein the scaffold composition: (i) is a porous scaffold material; and (b) administering an active agent into the scaffold, thereby locally transducing an immune cell (e.g., a hematopoietic stem cell (HSC) and/or a hematopoietic progenitor cell (HPC)) in the subject.

In another aspect, the present invention provides a method of genetically engineering an immune cell (e.g., a hematopoietic stem cell (HSC) and/or a hematopoietic progenitor cell (HPC)) in a subject in need thereof, comprising: (a) administering to a subject a scaffold composition, thereby recruiting immune cells (e.g., hematopoietic stem cells (HSCs) and/or hematopoietic progenitor cells (HPCs)) to form a bone marrow niche, wherein the scaffold composition: (i) is a porous scaffold material; and (b) administering an active agent into the scaffold, thereby genetically engineering an immune cell (e.g., a hematopoietic stem cell (HSC) and/or a hematopoietic progenitor cell (HPC)) in the subject.

In another aspect, the present invention provides a method of promoting the regeneration of immune cells (e.g., T cells) in a subject in need thereof, comprising: (a) administering to a subject a scaffold composition, wherein the scaffold composition: (i) is a porous scaffold material; and (b) administering an active agent into the scaffold, thereby promoting the regeneration of immune cells (e.g., T cells).

In various embodiments of the above aspects or any other aspect of the invention delineated herein, the scaffold composition recruits immune cells (e.g., hematopoietic stem cells (HSCs) and/or hematopoietic progenitor cells (HPCs)) to form a bone marrow niche in situ. In one embodiment, the method results in the local transduction of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 99% or more of hematopoietic progenitor cells (HPCs) and/or hematopoietic stem cells (HSCs) recruited to the scaffold composition in situ. In another embodiment, the method results in the local transduction of between about 1% and about 50% of hematopoietic progenitor cells (HPCs) and/or hematopoietic stem cells (HSCs) recruited to the scaffold composition in situ. In another embodiment, the method results in the local transduction of between about 1% and about 25% of hematopoietic progenitor cells (HPCs) and/or hematopoietic stem cells (HSCs) recruited to the scaffold composition in situ. In another embodiment, the method results in the local transduction of between about 25% and about 50% of hematopoietic progenitor cells (HPCs) and/or hematopoietic stem cells (HSCs) recruited to the scaffold composition in situ.

In various embodiments of the above aspects or any other aspect of the invention delineated herein, the active agent comprises an amino acid, a peptide, a protein, a nucleic acid, an oligonucleotide, a vector, a small molecule, or a combination thereof. In some embodiments, the active agent comprises a composition for modifying cells, optionally wherein the composition for modifying cells comprises a system for generating site-specific gene modifications. In some embodiments, the active agent comprises a CRISPER/Cas9 system, an RNAi, a transcription activator-like effector nuclease (TALEN), a transcription activator-like effectors (TALEs), a meganuclease, a zinc finger nucleases (ZFN), or a combination thereof.

In some embodiments, the active agent comprises a gene therapy and/or a chimeric antigen receptor (CAR) T-cell therapy. In some embodiments, the active agent comprises a nucleic acid. In some embodiments, the active agent comprises a vector. In some embodiments, the active agent comprises an expression vector. In some embodiments, the active agent comprises a viral vector. In some embodiments, the active agent comprises a viral vector selected from the group consisting of a retrovirus vector, a herpes simplex vector, a lentivirus vector, an adenovirus vector, and an adeno-associated virus vector. In some embodiments, the active agent comprises a lentivirus vector. In some embodiments, the active agent comprises a nucleic acid sequence encoding a gene product.

In various embodiments of the above aspects or any other aspect of the invention delineated herein, the method results in modification of a hematopoietic stem cells (HSC) and/or a hematopoietic progenitor cells (HPC) recruited to the scaffold composition in situ to replace a polynucleotide or gene product, or to add or knockdown a gene product.

In various embodiments of the above aspects or any other aspect of the invention delineated herein, the scaffold material is a hydrogel. In some embodiments, the scaffold material is a cryogel. In some embodiments, the scaffold material comprises a polymer or co-polymer selected from the group consisting of polylactic acid, polyglycolic acid, PLGA, alginate or an alginate derivative, gelatin, collagen, fibrin, agarose, hyaluronic acid, poly(lysine), polyhydroxybutyrate, poly-epsilon-caprolactone, polyphosphazines, poly(vinyl alcohol), poly(alkylene oxide), poly(ethylene oxide), poly(allylamine), poly(acrylate), poly(4-aminomethylstyrene), pluronic polyol, polyoxamer, poly(uronic acid), poly(anhydride), poly(vinylpyrrolidone), and any combination thereof. In some embodiments, the scaffold material comprises a polymer or co-polymer selected from the group consisting of alginate, alginate derivative, and any combination thereof. In some embodiments, the scaffold material comprises alginate. In some embodiments, the scaffold material comprises methacrylated alginate (MA-alginate). In some embodiments, the scaffold material comprises anionic alginate. In some embodiments, the scaffold material comprises a polymer or co-polymer selected from the group consisting of hyaluronic acid, hyaluronic acid derivative, and any combination thereof. In some embodiments, the scaffold material comprises a hyaluronic acid or a hyaluronic acid-derivative. In some embodiments, the scaffold material comprises a click-hydrogel or a click cryogel. In some embodiments, the scaffold material comprises a click-alginate, a click gelatin, or a click hyaluronic acid.

In various embodiments of the above aspects or any other aspect of the invention delineated herein, the scaffold material comprises pores having a diameter between about 1 nm and about 100 μm. In some embodiments, the scaffold material comprises pores having a diameter between about 1 μm and about 100 μm. In some embodiments, the scaffold material comprises a macropore. In some embodiments, the macropore has a diameter between about 20 μm and about 80 μm. In some embodiments, the macropore has a diameter between about 50 μm and about 80 μm. In some embodiments, the scaffold material comprises macropores of different sizes.

In various embodiments of the above aspects or any other aspect of the invention delineated herein, the scaffold composition is implantable and/or injectable.

In various embodiments of the above aspects or any other aspect of the invention delineated herein, the scaffold composition further comprises a growth factor. In some embodiments, the growth factor comprises a bone morphogenetic protein (BMP). In some embodiments, the growth factor is selected from the group consisting of a BMP-2, a BMP-4, a BMP-6, a BMP-7, a BMP-12, a BMP-14, and combinations thereof. In some embodiments, the growth factor comprises a BMP-2. In some embodiments, the growth factor is encapsulated in the scaffold material. In some embodiments, the growth factor is released from the scaffold material over about 7-30 days.

In various embodiments of the above aspects or any other aspect of the invention delineated herein, the scaffold composition further comprises a differentiation factor. In some embodiments, the differentiation factor binds to a Notch receptor. In some embodiments, the Notch receptor is selected from the group consisting of a Notch-1 receptor, a Notch-2 receptor, a Notch-3 receptor, a Notch-4 receptor, and any combination thereof. In some embodiments, the differentiation factor is selected from the group consisting of a Delta-like 1 (DLL-1), a Delta-like 2 (DLL-2), a Delta-like 3 (DLL-3), a Delta-like 4 (DLL-4), a Jagged 1, a Jagged 2, and any combination thereof. In some embodiments, the differentiation factor is bound, directly or indirectly, to the scaffold material. In some embodiments, the differentiation factor is covalently bound to the scaffold material or covalently bound to a tether that is covalently bound to the scaffold material.

In various embodiments of the above aspects or any other aspect of the invention delineated herein, the scaffold composition further comprises a cytokine. In some embodiments, the cytokine comprises interleukin-7 (IL-7). In some embodiments, the cytokine is encapsulated in the scaffold material. In some embodiments, the cytokine is released from the scaffold material over about 7-30 days.

In various embodiments of the above aspects or any other aspect of the invention delineated herein, the scaffold composition further comprises a homing factor. In some embodiments, the homing factor comprises a stem cell differentiation factor (SDF-1). In some embodiments, the homing factor is encapsulated in the scaffold material. In some embodiments, the homing factor is released from the scaffold material over about 7-30 days.

In various embodiments of the above aspects or any other aspect of the invention delineated herein, wherein: (i) the growth factor promotes formation of tissue on or around the administered scaffold material to form a bone marrow niche; (ii) the differentiation factor promotes the differentiation of a stem cell to a lymphoid lineage cell; and/or (iii) the homing factor promotes the infiltration of a stem cell and/or a progenitor cell to the bone marrow niche.

In some embodiments, the stem cell is a transplanted stem cell. In some embodiments, the stem cell is a hematopoietic stem cell.

In various embodiments of the above aspects or any other aspect of the invention delineated herein, the wherein the method results in (i) transduction of an immune cell localized in the scaffold material in vivo, optionally wherein the immune cell comprises a hematopoietic stem cell (HSC), optionally wherein the HSC comprises a myeloid and/or a lymphoid hematopoietic cell; (ii) greater transduction of an immune cell, optionally a primitive Lin− Kit+ Sca+ hematopoietic cell (HSC), localized in the scaffold material in vivo as compared to interfemoral injection of the same dose of active agent, optionally, by at least about 5%, or, at least about 10%, or at least about 20%, or at least about 30%, or at least about 40%, or at least about 50%, or at least about 60%, or at least about 70%, or at least about 80%, or at least about 90%, or at least about 95%, or at least about 99%, or up to and including a 100% increase, or any increase between about 5 and about 100%; (iii) recruitment of endogenous and/or transplanted immune cells, optionally hematopoietic stem cells (HSC), to the scaffold material within about 1-3 weeks after administration; and/or (iv) an increase in the number of immune cells, optionally T-cell competent progenitor cells, that traffic to the thymus to enhance immune reconstitution. In various embodiments of the above aspects or any other aspect of the invention delineated herein, the subject is a human.

In various embodiments of the above aspects or any other aspect of the invention delineated herein, the composition is administered to the subject via injection, optionally, intravenously, intramuscularly, or subcutaneously. In some embodiments, the scaffold is administered to the subclavicular fossa of the subject.

In various embodiments of the above aspects or any other aspect of the invention delineated herein, the subject has or is receiving a hematopoietic stem cell transplantation (HSCT). In some embodiments, the administration of the composition is prior to, concurrently with, or subsequent to the HSCT.

In various embodiments of the above aspects or any other aspect of the invention delineated herein, the subject has not received a hematopoietic stem cell transplantation (HSCT).

In certain embodiments of the above aspects or any other aspect of the invention delineated herein, the scaffold composition is cell free prior to administration to the subject.

In certain embodiments of the above aspects or any other aspect of the invention delineated herein, the immune cell comprises (i) a stem cell and/or progenitor cell, optionally wherein the stem cell and/or the progenitor cell comprises a hematopoietic stem cell (HSC) and/or a hematopoietic progenitor cell (HPC); (ii) a lymphocyte, optionally wherein the lymphocyte comprises a T cell, a B cell, and/or a natural killer (NK) cell; (iii) a myeloid-derived cell, optionally wherein the myeloid-derived cell comprises a neutrophil, an eosinophil, a basophil, a monocyte, a macrophage, and/or a dendritic cell; (iv) a modified immune cell; and/or (v) any combination thereof.

In various embodiments of the above aspects or any other aspect of the invention delineated herein, further comprising administering an additional dose of the active agent into the scaffold. In some embodiments, the method further comprises administering an additional active agent into the scaffold.

### BRIEF DESCRIPTION OF THE DRAWINGS

FIG. 1A shows flow analysis of the scaffold compositions, also referred to herein as a cellular composition of cell-free biomaterial-based bone marrow cryogels (BMC), 21 days after administration in comparison with endogenous bone marrow. The percentage of the indicated hematopoietic cell subpopulations, including B220, CD11b, CD3, Lin− Kit+, and Lin− Kit+ Sca+ cells, are shown.

FIG. 1B shows the percentage of transduction in the indicated hematopoietic subpopulations, including B220, CD11b, CD3, Lin− Kit+, and Lin− Kit+ Sca+ cells, 4 days after injection into the scaffold composition of VSV-G pseudotyped lentiviral vector.

FIG. 1C shows representative flow plots showing GFP expression from the transduced cells within the scaffold composition.

FIG. 1D shows fold transduction of primitive Lin− Kit+ Sca+ HSC-enriched fraction comparing intrafemural injection and localized scaffold composition injection of the same dose of lentivirus vector.

### DETAILED DESCRIPTION

### I. Definitions

In order that the present invention may be more readily understood, certain terms are first defined.

Unless otherwise defined herein, scientific and technical terms used in connection with the present invention shall have the meanings that are commonly understood by those of ordinary skill in the art. The meaning and scope of the terms should be clear, however, in the event of any latent ambiguity, definitions provided herein take precedent over any dictionary or extrinsic definition.

The use of the terms “a” and “an” and “the” and similar referents in the context of describing the invention (especially in the context of the following claims) are to be construed to cover both the singular and the plural (i.e., one or more), unless otherwise indicated herein or clearly contradicted by context. The terms “comprising, “having,” “including,” and “containing” are to be construed as open-ended terms (i.e., meaning “including, but not limited to”) unless otherwise noted. Recitation of ranges of values herein are merely intended to serve as a shorthand method of referring individually to each separate value recited or falling within the range, unless otherwise indicated herein, and each separate value is incorporated into the specification as if it were individually recited.

The term “about” or “approximately” usually means within 5%, or more preferably within 1%, of a given value or range.

Generally, the term “treatment” or “treating” is defined as the application or administration of a therapeutic agent to a patient, or application or administration of a therapeutic agent to an isolated tissue or cell line from a patient, said patient having a disease, a symptom of disease or a predisposition toward a disease, with the purpose to cure, heal, alleviate, relieve, alter, remedy, ameliorate, improve or affect the disease, the symptoms of disease or the predisposition toward disease. Thus, treating can include suppressing, inhibiting, preventing, treating, or a combination thereof. Treating refers, inter alia, to increasing time to sustained progression, expediting remission, inducing remission, augmenting remission, speeding recovery, increasing efficacy of or decreasing resistance to alternative therapeutics, or a combination thereof. “Suppressing” or “inhibiting”, refers, inter alia, to delaying the onset of symptoms, preventing relapse to a disease, decreasing the number or frequency of relapse episodes, increasing latency between symptomatic episodes, reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof. In one embodiment the symptoms are primary, while in another embodiment, symptoms are secondary. “Primary” refers to a symptom that is a direct result of a disorder, e.g., diabetes, while, secondary refers to a symptom that is derived from or consequent to a primary cause. Symptoms may be any manifestation of a disease or pathological condition.

Accordingly, as used herein, the term “treatment” or “treating” includes any administration of a composition described herein and includes: (i) preventing the disease from occurring in a subject which may be predisposed to the disease but does not yet experience or display the pathology or symptomatology of the disease; (ii) inhibiting the disease in an subject that is experiencing or displaying the pathology or symptomatology of the diseased (i.e., arresting further development of the pathology and/or symptomatology); or (iii) ameliorating the disease in a subject that is experiencing or displaying the pathology or symptomatology of the diseased (i.e., reversing the pathology and/or symptomatology).

By “treatment”, “prevention” or “amelioration” of a disease or disorder is meant delaying or preventing the onset of such a disease or disorder, reversing, alleviating, ameliorating, inhibiting, slowing down or stopping the progression, aggravation or deterioration the progression or severity of a condition associated with such a disease or disorder. In one embodiment, the symptoms of a disease or disorder are alleviated by at least 5%, at least 10%, at least 20%, at least 30%, at least 40%, or at least 50%.

Efficacy of treatment is determined in association with any known method for diagnosing the disorder. Alleviation of one or more symptoms of the disorder indicates that the composition confers a clinical benefit. Any of the therapeutic methods described to above can be applied to any suitable subject including, for example, mammals such as dogs, cats, cows, horses, rabbits, monkeys, and most preferably, humans.

As used herein, the term “subject” includes any subject who may benefit from being administered a hydrogel or an implantable drug delivery device of the invention. The term “subject” includes animals, e.g., vertebrates, amphibians, fish, mammals, non-human animals, including humans and primates, such as chimpanzees, monkeys and the like. In one embodiment of the invention, the subject is a human.

The term “subject” also includes agriculturally productive livestock, for example, cattle, sheep, goats, horses, pigs, donkeys, camels, buffalo, rabbits, chickens, turkeys, ducks, geese and bees; and domestic pets, for example, dogs, cats, caged birds and aquarium fish, and also so-called test animals, for example, hamsters, guinea pigs, rats and mice.

In certain embodiments, a subject can be one who has been previously diagnosed with or otherwise identified as suffering from or having a condition, disease, or disorder. A “subject in need” of treatment for a particular condition can be a subject having that condition, diagnosed as having that condition, or at increased risk of developing that condition relative to a given reference population. In some embodiments, the methods of treatment described herein comprise selecting a subject diagnosed with, suspected of having, or at risk of developing a hematological malignancy or being immunocompromised. In some embodiments, the methods described herein comprise selecting a subject diagnosed with, suspected of having, or at risk of developing a non-malignant disease, for example a non-malignant disease described herein.

As used herein, the term “administering,” for example, in the context of “administering to a subject a scaffold composition,” generally refers to the placement of the compositions described herein into a subject. In various embodiments, the compositions disclosed herein are administered to the subject via injection, optionally, intravenously, intramuscularly, or subcutaneously. In some embodiments, the compositions disclosed herein are administered to the subclavicular fossa of the subject. In some embodiments, the compositions disclosed herein are administered to multiple sites of the subject, for example, prior to undergoing a stem cell transplant. In certain embodiments, the compositions disclosed herein are administered at a site near the lymphatic system of the subject (e.g., in proximity to one or more of the neck, groin, and underarms of the subject). In certain aspects, following reconstitution of the subject's immune system, the compositions disclosed herein are removed (e.g., by surgical excision).

As used herein, the term “administering,” for example, in the context of “administering an active agent into the scaffold composition,” generally refers to the delivery of an active agent into the scaffold compositions described herein in situ and/or ex vivo. In various embodiments, the active agents are administered into the scaffold compositions disclosed herein via injection, e.g., intra scaffold injection. In certain embodiments, an active agent, such as a gene therapy, can be administered into the scaffold compositions described herein in situ, e.g., to enable transduction of target cells, e.g., myeloid and/or lymphoid hematopoietic cells, localized in the scaffold composition. In certain embodiments, an active agent, such as a gene therapy, administered into the scaffold compositions described herein in situ can result in at least about a 2-fold (e.g., at least about a 2-fold, at least about a 3-fold, at least about a 4-fold, at least about a 5-fold, at least about a 6-fold, at least about a 7-fold, at least about a 8-fold, at least about a 9-fold, at least about a 10-fold, at least about a 11-fold, at least about a 12-fold, at least about a 13-fold, at least about a 14-fold, at least about a 15-fold, at least about a 16-fold, at least about a 17-fold, at least about a 18-fold, at least about a 19-fold, at least about a 20-fold, at least about a 21-fold, at least about a 22-fold, at least about a 23-fold, at least about a 24-fold, or at least about a 25-fold) higher transduction in the HSC compartment as compared to direct intra-femoral delivery in the bone marrow.

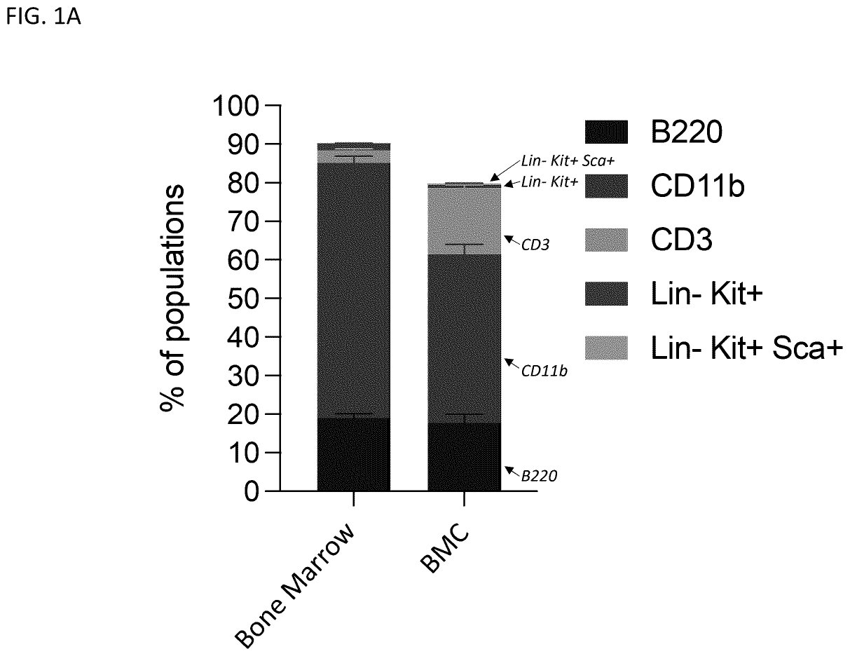

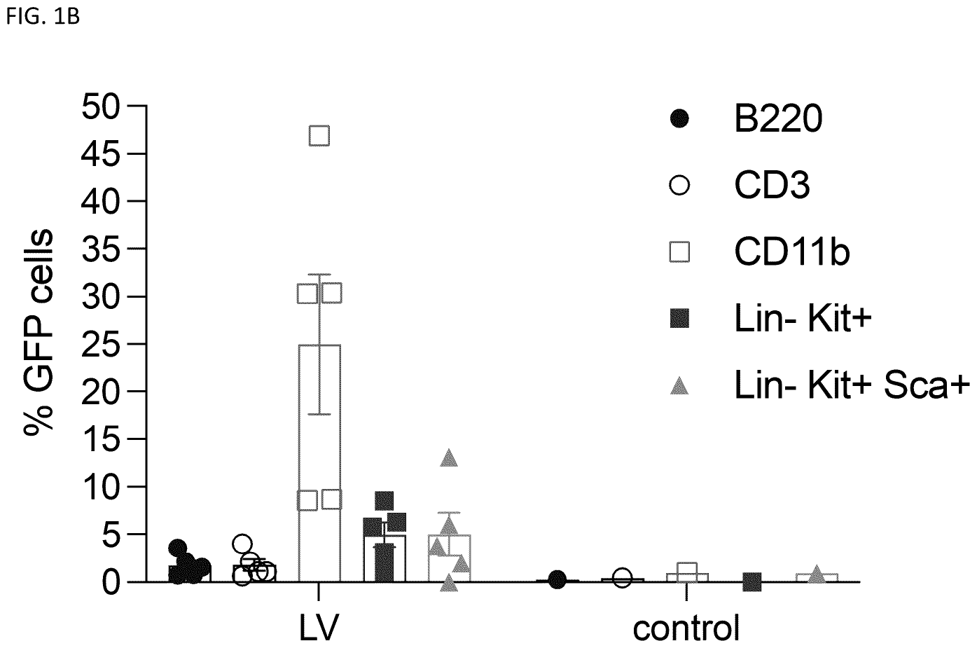

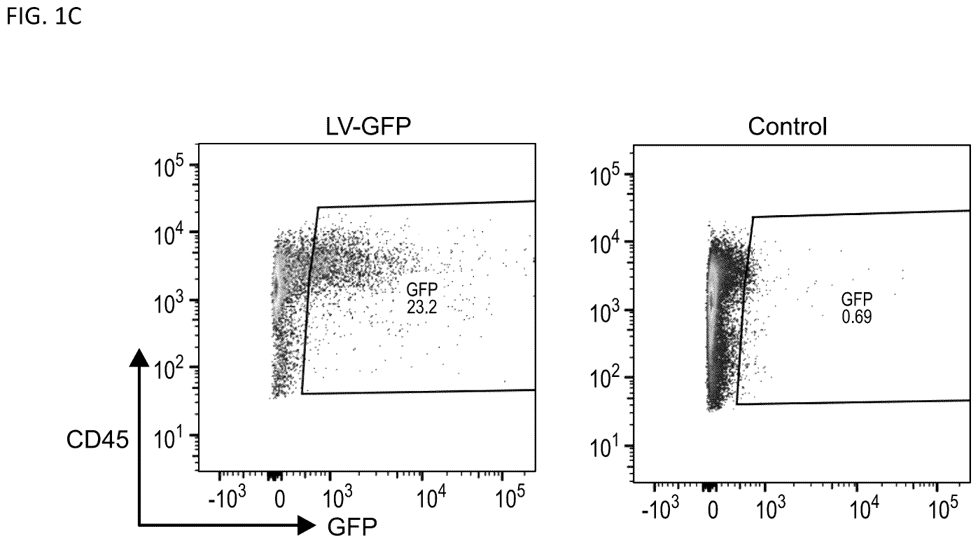

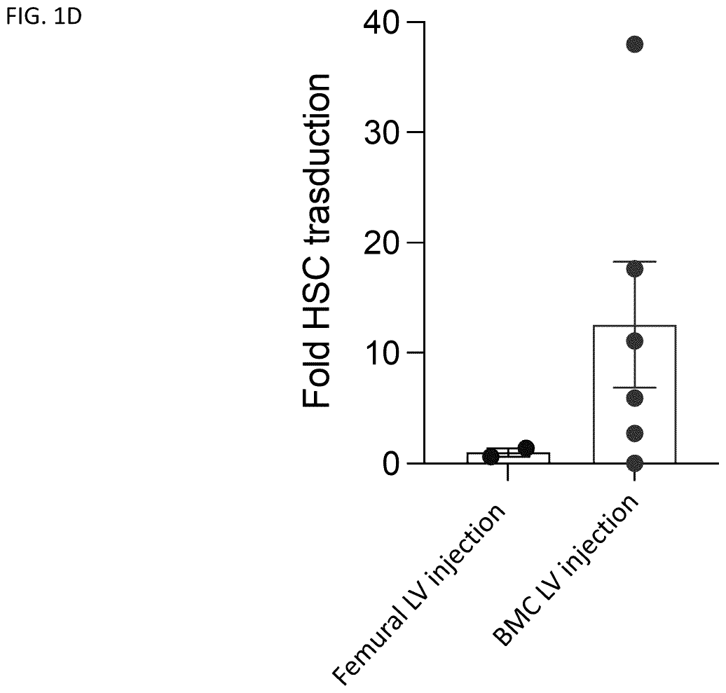

In certain embodiments, the compositions and methods disclosed herein are useful for the treatment of any disorder, disease, condition, or complication, for example, in which transplantation of hematopoietic stem cells and/or progenitor cells is desirable. However, in some embodiments, the scaffold materials disclosed herein can be used, even in absence of transplantation, to recruit endogenous immune cells (e.g., hematopoietic stem cells (HSCs) and/or hematopoietic progenitor cells (HPCs)), and to rebuild a functional bone marrow niche with physical properties and cellular composition similar to the endogenous bone marrow (FIG. 1A).

In certain embodiments, the compositions and methods disclosed herein are useful for the treatment of any disorder, disease, condition, or complication in which the modification of a cell, such as an immune cell (e.g., a hematopoietic stem cell (HSC) and/or a hematopoietic progenitor cell (HPC)) is desirable. In certain embodiments, such modifications of a cell may include, without limitation, gene therapy and/or chimeric antigen receptor (CAR) T-cell therapy, by any means known in the art.

In certain embodiments, a gene therapy may comprise the administration of an active agent, such as a nucleic acid and/or a protein (either native or modified), into a subject's cells according to a method described herein. Gene therapies may be used to supply a functional copy of a mutated gene, inactivate a gene by targeting the gene's DNA directly or targeting the mRNA transcript from the gene, or introduce a new gene into a target cell. Exemplary gene therapies that may be used in the methods described herein include, without limitation, (1) tumor suppressor gene therapies that restore cell control through replacing tumor suppressor genes (e.g., p53 and IAWO); (2) oncogene inhibition therapies that inactivate dominant oncogenes (e.g., EGFR and CLDN3); (3) suicide gene therapies by an enzyme/prodrug system (e.g., herpes simplex virus-thymidine kinase (HVS-TK) system) or activating expression of a toxin (e.g., diphtheria toxin-A); (4) antiangiogenic gene therapy by delivering VEGFRs or angiogenesis inhibitors (e.g., angiostatin and endostatin); (5) immunopotentiation gene therapies by strengthening the immune response to tumor cells (e.g., chimeric antigen receptor T cell therapy), augmenting the expression of tumor antigens or the production of cytokines, interleukins (e.g., IL-21) and growth factors; (6) multi-drug resistance (MDR) associated gene therapies to knockdown genes such as MDR1 and surviving; and/or (7) oncolytic virotherapies that utilize oncolytic viruses to preferentially kill tumor cells (e.g., vesicular stomatitis virus). Other gene therapies suitable for use in the compositions and methods disclosed herein are known in the art.

In certain embodiments, a gene therapy replaces a faulty gene or adds a new gene in an attempt to cure a disease and/or improve a subject's ability to fight a disease. Gene therapy may be used to treat a wide range of diseases, including, without limitation, cancer, hematological malignancies, non-malignant hematological diseases, genetic immune deficiencies, cystic fibrosis, heart disease, diabetes, hemophilia, and AIDS.

Examples of such disorders include hematological malignancies and non-malignant hematological diseases. The compositions and methods disclosed herein are also useful for reconstituting T-cells and B-cells and accordingly can be broadly applied to other diseases in which immunodeficiency is implicated, such as age-related vaccine failure, autoimmune disorders (e.g., rheumatoid arthritis and diabetes), infectious disease, and others. In some embodiments, the disease is a stem cell disorder or a progenitor cell disorder. In some embodiments, the disease is a hematological malignancy. Exemplary hematological malignancies which can be treated with the compositions and methods described herein include, but are not limited to, acute lymphoid leukemia, acute myeloid leukemia, chronic lymphoid leukemia, chronic myeloid leukemia, diffuse large B-cell non-Hodgkin's lymphoma, mantle cell lymphoma, lymphoblastic lymphoma, Burkitt's lymphoma, follicular B-cell non-Hodgkin's lymphoma, T-cell non-Hodgkin's lymphoma, lymphocyte predominant nodular Hodgkin's lymphoma, multiple myeloma, and juvenile myelomonocytic leukemia.

In some embodiments, the disease is a non-malignant disorder. Exemplary non-malignant diseases which can be treated with the compositions and methods described herein include, but are not limited to, myelofibrosis, myelodysplastic syndrome, amyloidosis, severe aplastic anemia, paroxysmal nocturnal hemoglobinuria, immune cytopenias, systemic sclerosis, rheumatoid arthritis, multiple sclerosis, systemic lupus erythematosus, Crohn's disease, chronic inflammatory demyelinating polyradiculoneuropathy, human immunodeficiency virus (HIV), Fanconi anemia, sickle cell disease, beta thalassemia major, Hurler's syndrome (MPS-IH), adrenoleukodystrophy, metachromatic leukodystrophy, familial erythrophagocytic lymphohistiocytosis and other histiocytic disorders, severe combined immunodeficiency (SCID), and Wiskott-Aldrich syndrome.

As used herein, the term “immune cells” generally refer to resting and/or activated cells of the immune system involved in defending a subject against both infectious disease and foreign materials. Examples of immune cells include, without limitations, white blood cells including, e.g., neutrophils, eosinophils, basophils, lymphocytes (e.g., B-cells, T-cells, and natural killer cells), monocytes, macrophages (including, e.g., resident macrophages, resting macrophages, and activated macrophages); as well as Kupffer cells, histiocytes, dendritic cells, Langerhans cells, mast cells, microglia, and any combinations thereof. In some embodiment, immune cells include derived immune cells, for example, immune cells derived from lymphoid stem cells and/or myeloid stem cells. In some embodiment, immune cells include white blood cells (leukocytes) which are derived from hematopoietic stem cells (HSC) and/or hematopoietic progenitor cells (HPC). In some embodiment, immune cells include hematopoietic stem cells (HSC) and/or hematopoietic progenitor cells (HPC). In some embodiment, immune cells include lymphocytes (T cells, B cells, natural killer (NK) cells) and/or myeloid-derived cells (neutrophil, eosinophil, basophil, monocyte, macrophage, dendritic cells). As used herein, the term “T cell” refers to all types of immune cells expressing CD3 including, without limitation, T-helper cells (CD4+ cells), cytotoxic T-cells (CD8+ cells), T-regulatory cells (Treg), and gamma-delta T cells. As used herein, the term “cytotoxic cell” refer, without limitation, to cells capable of mediating cytotoxicity responses, such as CD8+ T cells, natural-killer (NK) cells, and neutrophils. As used herein, the term “stem cell” generally includes pluripotent or multipotent stem cells. “Stem cells” includes, e.g., embryonic stem cells (ES); mesenchymal stem cells (MSC); induced-pluripotent stem cells (iPS); and committed progenitor cells (hematopoietic stem cells (HSC); bone marrow derived cells, neural progenitor cells, etc.).

By the term “modified” as used herein, is meant a changed state or structure of a molecule or cell of the disclosure. Molecules may be modified in many ways, including chemically, structurally, and functionally. Cells may be modified, for example, through the introduction of nucleic acids and/or proteins.

The terms “modified cell” or “engineered cell” refer interchangeably to a genetically altered (e.g., transduced, transformed, transfected, and/or conjugated) cell. The term refers to the particular subject cell and also to the progeny or potential progeny of such a cell. Because certain modifications may occur in succeeding generations due to either mutation or environmental influences, such progeny may not, in fact, be identical to the parent cell, but are still included within the scope of the term as used herein. In certain embodiments, a nucleic acid (e.g., a vector) and/or a polypeptide (e.g., a protein and/or a peptide) may be introduced (e.g., transduced, transformed, transfected, and/or conjugated) into a cell, for example, according to the methods described herein. Once a nucleic acid and/or a polypeptide has been introduced into the cell, it may be referred to as a “modified cell” herein. Once the nucleic acid molecule or vector is introduced into the subject's cell, the resultant modified cell should be capable of expressing an encoded polypeptide and, e.g., correctly localizing the encoded polypeptide for its intended function, e.g., transporting the encoded polypeptide to the cell surface).

As used herein, the term “transduction” refers to the process of introducing a nucleic acid molecule (e.g., comprising an exogenous nucleic acid molecule, e.g., a transgene) into a cell via a virus and/or a viral vector. The nucleic acid molecule can be, without limitation, a vector, DNA, RNA, and/or a plasmid. The term “transduction” is may be used to describe the introduction of recombinant viral vector particles into target cells (e.g., host cells). The virus or viral vector may comprise, for example, DNA or RNA, double-stranded or single-stranded, monopartite or multipartite, linear or circular viral nucleic acids. Transduction of cells can be either in vitro, ex vivo, in vivo, and/or in situ. Transgene expression can be transient or stable.

As used herein, the term “transduction efficiency” refers to the ability of a virus and/or a viral vector to transduce cells (e.g., the ability that a polynucleotide or nucleic acid molecule can be introduced into a host cell by the virus and/or viral vector), and more particularly the efficiency with which a virus and/or a viral vector transduces host cells. In particular embodiment, the transduction efficiency is in vivo transduction efficiency, and refers to the ability of a virus and/or a viral vector to transduce host cells in vivo following administration of the virus and/or the viral vector to the subject. In some embodiments, the virus and/or the viral vector can be administered into the scaffold compositions described herein in situ. Transduction efficiency can be assessed in a number of ways known in the art, including, e.g., assessing the number of host cells transduced following exposure to, or administration of, a given number of vector particles (e.g., as assessed by expression of a reporter gene from the vector genome, such as GFP or eGFP, using microscopy or flow cytometry techniques); the amount of vector DNA (e.g., number of vector genomes) in a population of host cells following exposure to a given number of vector particles; the amount of vector RNA in population of host cells following exposure to a given number of vector particles; and/or the level of protein expression from a reporter gene (e.g., GFP or eGFP) in the vector genome in a population of host cells following exposure to, or administration of, a given number of vector particles. In one example, the transduction efficiency of a specific virus and/or viral vector against a specific cell type is determined by the percentage of cells of the specific cell type which have been detected positive for the presence of the specific virus and/or viral vector. In another example, the transduction efficiency of a specific virus and/or viral vector against a specific cell type is assessed by comparing the frequencies with which the presence of said specific virus and/or viral vector is detected in the cells of said specific cell type, against the frequencies with which the presence of another virus and/or viral vector is detected in the cells of said specific cell type.

As used herein, the term “transformation” refers to the uptake of nucleic acids from the environment, for example, by a bacterial cell.

As used herein, the term “transfection” refers to the process of introducing small molecules, such as nucleic acids and/or proteins, into cells, particularly mammalian cells, by non-viral methods. Transfection may be transient or stable. Common transfection methods include, without limitation, calcium phosphate, cationic polymers (such as PEI), magnetic beads, electroporation, and commercial lipid-based reagents such as Lipofectamine.

As used herein, the term “conjugate” or “conjugation” refers to the attachment of two or more entities to form one entity. For example, the methods of the present invention provide conjugation of a small molecule, including, without limitation, a nucleic acid and/or a protein, joined with another entity. The attachment can be by means of linkers, chemical modification, peptide linkers, chemical linkers, covalent or non-covalent bonds, or protein fusion or by any means known to one skilled in the art. The joining can be permanent or reversible. Methods for conjugation are well known by persons skilled in the art and are encompassed for use in the present invention. Various methods of transducing, transforming, transfecting, and/or conjugating cells with small molecules, such as nucleic acids and/or proteins, are known in the art, and may be used according to the methods described herein. A common way to validate that a nucleic acid and/or a protein was successfully introduced into cells is to measure protein expression, which is typically performed, for example, by Western blot or immunostaining.

As used herein, the term “T cell receptor” or “TCR” refers to a complex of membrane proteins that participate in the activation of T cells in response to the presentation of antigen. The TCR is responsible for recognizing antigens bound to major histocompatibility complex molecules. TCR is composed of a heterodimer of an alpha (α) and beta (β) chain, although in some cells the TCR consists of gamma and delta (γ/δ) chains. TCRs may exist in alpha/beta and gamma/delta forms, which are structurally similar but have distinct anatomical locations and functions. Each chain is composed of two extracellular domains, a variable and constant domain. In some embodiments, the TCR may be modified on any cell comprising a TCR, including, for example, a helper T cell, a cytotoxic T cell, a memory T cell, regulatory T cell, natural killer T cell, and gamma delta T cell.

As used herein, the term “hematopoietic stem cells” or “HSC” refers to stem cells that can differentiate into the hematopoietic lineage and give rise to all blood cell types such as white blood cells and red blood cells, including myeloid (e.g., monocytes and macrophages, neutrophils, basophils, eosinophils, erythrocytes, megakaryocytes/platelets, dendritic cells), and lymphoid lineages (e.g., T-cells, B-cells, K-cells). Stem cells are defined by their ability to form multiple cell types (multipotency) and their ability to self-renew. Hematopoietic stem cells can be identified, for example by cell surface markers such as CD34−, CD133+, CD48−, CD150+, CD244−, cKit+, Scal+, and lack of lineage markers (negative for B220, CD3, CD4, CD8, Macl, Grl, and Terl 19, among others).

As used herein, the term “hematopoietic progenitor cells” or “HPC” encompasses pluripotent cells which are committed to the hematopoietic cell lineage, generally do not self-renew, and are capable of differentiating into several cell types of the hematopoietic system, such as granulocytes, monocytes, erythrocytes, megakaryocytes, B-cells and T-cells, including, but not limited to, short term hematopoietic stem cells (ST-HSCs), multi-potent progenitor cells (MPPs), common myeloid progenitor cells (CMPs), granulocyte-monocyte progenitor cells (GMPs), megakaryocyte-erythrocyte progenitor cells (MEPs), and committed lymphoid progenitor cells (CLPs). The presence of hematopoietic progenitor cells can be determined functionally as colony forming unit cells (CFU-Cs) in complete methylcellulose assays, or phenotypically through the detection of cell surface markers (e.g., CD45−, CD34+, Terl 19−, CD16/32, CD127, cKit, Seal) using assays known to those of skill in the art.

As used herein, the term “vector” means any genetic construct, such as for example, a plasmid, phage, transposon, cosmid, chromosome, virus and/or virion, which is capable transferring nucleic acids between cells. Vectors may be capable of one or more of replication, expression, and insertion or integration, but need not possess each of these capabilities. Thus, the term includes cloning, expression, homologous recombination, and knock-out vectors. In certain aspects, prior to engraftment, a hematopoietic stem cells (HSC) and/or a hematopoietic progenitor cells (HPC) can be manipulated to express one or more desired polynucleotides or gene products (e.g., one or more of a polypeptide, amino acid sequence protein and/or enzyme). Gene therapy can be used to either modify a hematopoietic stem cells (HSC) and/or a hematopoietic progenitor cells (HPC) to replace a polynucleotide or gene product or to add or knockdown a gene product. In some embodiments, the genetic engineering is done, for example, to treat a disease and/or disorder. The disclosure contemplates methods of genetic engineering which encompass methods of locally transducing an immune cell (e.g., a hematopoietic stem cell (HSC) and/or a hematopoietic progenitor cell (HPC)) in a subject in need thereof, comprising: (a) administering to a subject a scaffold composition, thereby recruiting immune cells (e.g., hematopoietic stem cells (HSCs) and/or hematopoietic progenitor cells (HPCs)) to form a bone marrow niche, wherein the scaffold composition: (i) is a porous scaffold material; and (b) administering an active agent into the scaffold, thereby locally transducing an immune cell (e.g., a hematopoietic stem cell (HSC) and/or a hematopoietic progenitor cell (HPC)) in the subject.

In certain aspects of the present inventions, the immune cells (e.g., hematopoietic stem cells (HSCs) and/or hematopoietic progenitor cells (HPCs)) disclosed herein may be transduced in situ, for example, to express a polynucleotide (e.g., an exogenous polynucleotide) or a gene product (e.g., a transgene). For example, in certain embodiments, the immune cells (e.g., hematopoietic stem cells (HSCs) and/or hematopoietic progenitor cells (HPCs)) disclosed herein may be transduced with an expression vector to express a polynucleotide (e.g., an exogenous polynucleotide) or gene product (e.g., a transgene). In some embodiments, the hematopoietic stem cells (HSCs) and/or the hematopoietic progenitor cells (HPCs) recruited to the scaffold composition in situ is transduced with an expression vector to replace a polynucleotide or a gene product, or to add or knockdown a gene product. In some embodiments, the expression vector comprises a viral vector selected from the group consisting of a retrovirus vector, a herpes simplex vector, an adenovirus vector, a lentivirus vector, and an adeno-associated virus vector. The term “vector” may also sometimes refer to transport vehicles comprising the vector, such as viruses or virions, which are able to transfer the vector into and between host cells.

The term “genetically modified” or “engineered” cell as used herein refers to a cell into which an exogenous nucleic acid has been introduced (or a descendant of such a cell that has inherited at least a portion of the nucleic acid). The nucleic acid may for example contain a sequence that is exogenous to the cell, it may contain native sequences (i.e., sequences naturally found in the cells) but in a non-naturally occurring arrangement (e.g., a coding region linked to a promoter from a different gene), or altered versions of native sequences, etc. The process of transferring the nucleic into the cell can be achieved by any suitable technique. Suitable techniques include calcium phosphate or lipid-mediated transfection, electroporation, and transduction or infection using a viral vector. In some embodiments the polynucleotide or a portion thereof is integrated into the genome of the cell. The nucleic acid may have subsequently been removed or excised from the genome, provided that such removal or excision results in a detectable alteration in the cell relative to an unmodified but otherwise equivalent cell. In certain aspects, the disclosure provides methods of genetic engineering which comprise locally transducing an immune cell (e.g., a hematopoietic stem cell (HSC) and/or a hematopoietic progenitor cell (HPC)) in a subject in need thereof, comprising: (a) administering to a subject a scaffold composition, thereby recruiting immune cells (e.g., hematopoietic stem cells (HSCs) and/or hematopoietic progenitor cells (HPCs)) to form a bone marrow niche, wherein the scaffold composition: (i) is a porous scaffold material; and (b) administering an active agent into the scaffold, thereby locally transducing an immune cell (e.g., a hematopoietic stem cell (HSC) and/or a hematopoietic progenitor cell (HPC)) in the subject.

The term “gene delivery” or “gene transfer” refers to methods for introduction of exogenous nucleic acids, such as recombinant or foreign DNA, into host cells, such as immune cells (e.g., hematopoietic stem cells (HSCs) and/or hematopoietic progenitor cells (HPCs)). The transferred nucleic acids can remain non-integrated or integrate into the genome of the host cell. Gene delivery can take place, for example, by transduction, using non-viral based or viral based techniques, including, for example, vectors, or by transformation of cells, using known methods, such as electroporation and cell bombardment.

In some embodiments, the term “gene delivery” or “gene transfer” refers to the administration of an active agent, such as a composition for modifying cells, such as immune cells (e.g., hematopoietic stem cells (HSCs) and/or hematopoietic progenitor cells (HPCs)). In some embodiments, such a composition may comprise a CRISPER/Cas9 system, an RNAi (including but are not limited to, siRNA, shRNA, endogenous microRNA and artificial microRNA), a transcription activator-like effector nuclease (TALEN), a transcription activator-like effectors (TALEs), a meganuclease, a zinc finger nucleases (ZFN), or a combination thereof. In some embodiments, the active agent may comprises a vector, such as an expression vector and/or a viral vector. In some embodiments, the active agent may comprise a retrovirus vector, a herpes simplex vector, a lentivirus vector, an adenovirus vector, or an adeno-associated virus vector. In particular embodiments, the active agent comprises a lentivirus vector. In some embodiments, the active agent comprises a nucleic acid sequence encoding a gene product. In some embodiments, the active agent comprises a lipid nanoparticle (LNP). In some embodiments, the active agent comprises a gene therapy.

“Nucleic acid” includes any molecule composed of or comprising monomeric nucleotides. The term “nucleotide sequence” may be used interchangeably with “nucleic acid” herein. A nucleic acid may be an oligonucleotide or a polynucleotide. A nucleic acid may be a DNA or an RNA. A nucleic acid may be a gene. A nucleic acid may be chemically modified or artificial.

The term “transgene” refers to a gene that has been introduced into a host cell, e.g., immune cell (e.g., hematopoietic stem cell (HSC) and/or hematopoietic progenitor cell (HPC)). The transgene may comprise sequences that are native to the cell, sequences that do not occur naturally in the cell, or combinations thereof. A transgene may contain sequences coding for one or more proteins that may be operably linked to appropriate regulatory sequences for expression of the coding sequences in the cell.

The term “transduction”” refers to the delivery of a nucleic acid molecule into a recipient host cell, e.g., immune cell (e.g., hematopoietic stem cell (HSC) and/or hematopoietic progenitor cell (HPC)), such as by a gene delivery vector, such as a non-viral or viral vector.

The term “host cell” or “target cell” refers to the immune cell (e.g., the hematopoietic stem cell (HSC) and/or the hematopoietic progenitor cell (HPC)) into which the nucleic acid delivery takes place.

The term “reduced” or “reduce” or “decrease” as used herein generally means a decrease of at least 5%, for example a decrease by at least about 10%, or at least about 20%, or at least about 30%, or at least about 40%, or at least about 50%, or at least about 60%, or at least about 70%, or at least about 80%, or at least about 90% or up to and including a 100% decrease (i.e. substantially absent or below levels of detection), or any decrease between 5-100% as compared to a reference level, as that term is defined herein, and as determined by a method that achieves statistical significance (p<0.05).

The term “increased” or “increase” as used herein generally means an increase of at least 5%, for example an increase by at least about 10%, or at least about 20%, or at least about 30%, or at least about 40%, or at least about 50%, or at least about 60%, or at least about 70%, or at least about 80%, or at least about 90% or up to and including a 100% increase (i.e., substantially above levels of detection), or any increase between 5-100% as compared to a reference level, as that term is defined herein, and as determined by a method that achieves statistical significance (p<0.05). In some embodiments, the methods described herein can result in a greater transduction of immune cells (e.g., hematopoietic stem cells (HSCs) and/or hematopoietic progenitor cells (HPCs)), such as primitive Lin− Kit+ Sca+ hematopoietic cells (HSCs), localized in the scaffold material in vivo as compared to interfemoral injection of the same dose of active agent, optionally, by at least about 5%, or, at least about 10%, or at least about 20%, or at least about 30%, or at least about 40%, or at least about 50%, or at least about 60%, or at least about 70%, or at least about 80%, or at least about 90%, or at least about 95%, or at least about 99%, or up to and including a 100% increase, or any increase between about 5 and about 100%.

As used herein, the term “standard” or “reference” refers to a measured biological parameter including, but not limited to, the level (e.g., concentration) of a gene product in a known sample against which another sample is compared; alternatively, a standard can simply be a reference number that represents an amount of the measured biological parameter that defines a baseline for comparison. The reference number can be derived from either a sample taken from an individual, or a plurality of individuals or cells obtained therefrom. That is, the “standard” does not need to be a sample that is tested, but can be an accepted reference number or value. A series of standards can be developed that take into account an individual's status, e.g., with respect to age, gender, weight, height, ethnic background etc. A standard level can be obtained, for example, from a known sample from a different individual (e.g., not the individual being tested). A known sample can also be obtained by pooling samples from a plurality of individuals (or cells obtained therefrom) to produce a standard over an averaged population. Additionally, a standard can be synthesized such that a series of standards are used to quantify the biological parameter in an individual's sample. A sample from the individual to be tested can be obtained at an earlier time point (presumably prior to the onset of treatment) and serve as a standard or reference compared to a sample taken from the same individual after the onset of treatment. In such instances, the standard can provide a measure of the efficacy of treatment. Ranges provided herein are understood to be shorthand for all of the values within the range. For example, a range of 1 to 100 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100.

As used herein, the term “gene” has its meaning as understood in the art. It will be appreciated by those of ordinary skill in the art that the term “gene” may include gene regulatory sequences (e.g., promoters, enhancers, etc.) and/or intron sequences. It will further be appreciated that definitions of gene include references to nucleic acids that do not encode proteins but rather encode a functional RNA molecule including, but not limited to, an RNAi agent, a ribozyme, a tRNA, an mRNA, etc. For the purpose of clarity it should be noted that, as used in the present application, the term “gene” generally refers to a portion of a nucleic acid that encodes a protein; the term may optionally encompass regulatory sequences, as will be clear from context to those of ordinary skill in the art. This definition is not intended to exclude application of the term “gene” to non-protein-coding expression units but rather to clarify that, in most cases, the term as used in this document refers to a protein-coding nucleic acid.

As used herein, the term “active agent” refers to an active ingredient that is intended for use in a particular application. In some embodiments, the term “active agent” refers to an agent that possesses therapeutic, prophylactic, or diagnostic properties in vivo, for example when administered to a human subject or an animal, including mammals and domestic animals. Examples of active agents include, but are not limited to, amino acids, proteins, peptides, antibodies, growth factors, nucleic acids, vectors, sugars, antigens, vaccines, viruses, enzymes, cells, small molecules, drugs, and any combination thereof. In some embodiments, the term “active agent” refers to a composition for modifying cells, such as a composition comprising a system for generating site-specific gene modifications. In some embodiments, the active agent comprises a CRISPER/Cas9 system, an RNAi (including but are not limited to, siRNA, shRNA, endogenous microRNA and artificial microRNA), a transcription activator-like effector nuclease (TALEN), a transcription activator-like effectors (TALEs), a meganuclease, a zinc finger nucleases (ZFN), or a combination thereof. In some embodiments, the active agent comprises a vector. In some embodiments, the active agent comprises an expression vector. In some embodiments, the active agent comprises a viral vector, such as a retrovirus vector, a herpes simplex vector, a lentivirus vector, an adenovirus vector, or an adeno-associated virus vector. In particular embodiments, the active agent comprises a lentivirus vector. In some embodiments, the active agent comprises a nucleic acid sequence encoding a gene product. In some embodiments, the active agent comprises a lipid nanoparticle (LNP). In some embodiments, the active agent comprises a gene therapy.

### II. Compositions for Modulating Immune System

The present invention features compositions and methods that modulate the immune system of a subject. The compositions disclosed herein comprise one or more scaffold materials (e.g., a porous scaffold material), which may be administered to or otherwise implanted or injected into a subject. For example, in certain embodiments the compositions disclosed herein comprise a growth factor and a differentiation factor, that may be loaded into or encapsulated by a scaffold material and, following its administration to a subject, such composition promotes the formation of tissue (e.g., bone tissue) on or around the administered scaffold material to form a bone marrow niche.

Accordingly, in certain aspects the scaffold materials disclosed herein provide a delivery vehicle for one or more growth factors, differentiation factors, homing factors, cytokines, chemokines and any other active agents.

In certain aspects, the scaffold materials disclosed herein can be used to enable localized transduction of immune cells (e.g., hematopoietic stem cells (HSCs) and/or hematopoietic progenitor cells (HPCs)) recruited to the scaffold composition in situ. In some embodiments, the scaffold materials disclosed herein can be used, even in absence of transplantation, to recruit endogenous immune cells (e.g., hematopoietic stem cells (HSCs) and/or hematopoietic progenitor cells (HPCs)), and to rebuild a functional bone marrow niche with physical properties and cellular composition similar to the endogenous bone marrow (FIG. 1A).

Direct injection of viral and non-viral agents into the scaffold compositions has several advantages over systemic intravenous administration, including, for example a reduction of the vector dose needed for effective hematopoietic progenitor cells (HPCs) and/or hematopoietic stem cells (HSCs) transduction; and reduction of innate and adaptive immune responses. For example, the methods of the present invention have been found useful for reducing very pronounced inflammatory storms associated with systemic intravenous administration of viral particles and that often require pre-treatment with immunosuppressive drugs.

Additionally, the scaffold compositions described herein provide a high portability of treatment. A practical aspect of the scaffold composition treatment is that it can be administered without hospitalization, can be cryopreserved, and requires no ex vivo culturing. Moreover, the scaffold compositions are not patient specific, and the same scaffold compositions can be used for all subjects. Therefore, in certain embodiments, the scaffold compositions provided herein can serve as an off the shelf reagent administered by simple subcutaneous injection. In certain embodiments, the scaffold compositions can be injected into the sub-clavicular space of a subject, similar to where other devices such as vascular ports or pacemakers can be located without significant morbidity or discomfort for subjects.

### Scaffolds

The composition of the present invention comprise a scaffold, e.g., a polymer scaffold. The scaffold can comprise one or more biomaterials. Preferably, the biomaterial is a biocompatible material that is non-toxic and/or non-immunogenic. As used herein, the term “biocompatible material” refers to any material that does not induce a significant immune response or deleterious tissue reaction, e.g., toxic reaction or significant irritation, over time when implanted into or placed adjacent to the biological tissue of a subject.

The scaffold can comprise biomaterials that are non-biodegradable or biodegradable. In certain embodiments, the biomaterial can be a non-biodegradable material. Exemplary non-biodegradable materials include, but are not limited to, metal, plastic polymer, or silk polymer. In certain embodiments, the polymer scaffold comprises a biodegradable material. The biodegradable material may be degraded by physical or chemical action, e.g., level of hydration, heat, oxidation, or ion exchange or by cellular action, e.g., elaboration of enzyme, peptides, or other compounds by nearby or resident cells. In certain embodiments, the polymer scaffold comprises both non-degradable and degradable materials.

In some embodiments, the scaffold composition can degrade at a predetermined rate based on a physical parameter selected from the group consisting of temperature, pH, hydration status, and porosity, the cross-link density, type, and chemistry or the susceptibility of main chain linkages to degradation. Alternatively, the scaffold composition degrades at a predetermined rate based on a ratio of chemical polymers. For example, a high molecular weight polymer comprised of solely lactide degrades over a period of years, e.g., 1-2 years, while a low molecular weight polymer comprised of a 50:50 mixture of lactide and glycolide degrades in a matter of weeks, e.g., 1, 2, 3, 4, 6, or 10 weeks. A calcium cross-linked gels composed of high molecular weight, high guluronic acid alginate degrade over several months (1, 2, 4, 6, 8, 10, or 12 months) to years (1, 2, or 5 years) in vivo, while a gel comprised of low molecular weight alginate, and/or alginate that has been partially oxidized, will degrade in a matter of weeks.

In certain embodiments, one or more compounds or proteins (e.g., the growth factors, the differentiation factors, and the homing factors), disclosed herein, are covalently or non-covalently linked or attached to the scaffold composition. In various embodiments, one or more compounds or proteins disclosed herein is incorporated on, into, or present within the structure or pores of, the scaffold composition.

In some embodiments, the scaffolds comprise biomaterials that are modified, e.g., oxidized or reduced. The degree of modification, such as oxidation, can be varied from about 1% to about 100%. As used herein, the degree of modification means the molar percentage of the sites on the biomaterial that are modified with a functional group. For example, the degree of modification can be about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%. It is intended that values and ranges intermediate to the recited values are part of this invention. Exemplary modified biomaterials, e.g., hydrogels, include, but not limited to, reduced-alginate, oxidized alginate, MA-alginate (methacrylated alginate) or MA-gelatin.

Exemplary biomaterials suitable for use as scaffolds in the present invention include glycosaminoglycan, silk, fibrin, MATRIGEL®, poly-ethyleneglycol (PEG), polyhydroxy ethyl methacrylate, polyacrylamide, poly(N-vinyl pyrolidone), (PGA), poly lactic-co-glycolic acid (PLGA), poly e-caprolactone (PCL), polyethylene oxide, poly propylene fumarate (PPF), poly acrylic acid (PAA), polyhydroxybutyric acid, hydrolysed polyacrylonitrile, polymethacrylic acid, polyethylene amine, esters of alginic acid; pectinic acid; and alginate, fully or partially oxidized alginate, hyaluronic acid, carboxy methyl cellulose, heparin, heparin sulfate, chitosan, carboxymethyl chitosan, chitin, pullulan, gellan, xanthan, collagen, gelatin, carboxymethyl starch, carboxymethyl dextran, chondroitin sulfate, cationic guar, cationic starch, and combinations thereof. In certain embodiments, the biomaterial is selected from the group consisting of alginate, fully or partially oxidized alginate, and combinations thereof.

The scaffolds of the present invention may comprise an external surface. Alternatively, or in addition, the scaffolds may comprise an internal surface. External or internal surfaces of the scaffolds of the present invention may be solid or porous. Pore size of the scaffolds can be less than about 10 nm, between about 100 nm-20 μm, or greater than about 20 μm, e.g., up to and including 1000 μm in diameter. For example, the pores may be nanoporous, microporous, or macroporous. For example, the diameter of nanopores is less than about 10 nm; the diameter of micropores is in the range of about 100 nm-20 μm; and, the diameter of macropores is greater than about 20 μm, e.g., greater than about 50 μm, e.g., greater than about 100 μm, e.g., greater than about 400 μm, e.g., greater than 600 μm or greater than 800 μm. In some embodiment the diameter of the pore is between about 20 μm and about 80 μm. In some embodiment the diameter of the pore is between about 50 μm and about 80 μm.

In some embodiments, the scaffolds of the present invention are organized in a variety of geometric shapes (e.g., discs, beads, pellets), niches, planar layers (e.g., thin sheets). For example, discs of about 0.1-200 millimeters in diameter, e.g., 5, 10, 20, 40, or 50 millimeters may be implanted subcutaneously. The disc may have a thickness of 0.1 to 10 millimeters, e.g., 1, 2, or 5 millimeters. The discs are readily compressed or lyophilized for administration to a patient. An exemplary disc for subcutaneous administration has the following dimensions: 8 millimeters in diameter and 1 millimeter in thickness.

In some embodiments, the scaffolds may comprise multiple components and/or compartments. In certain embodiments, a multiple compartment device is assembled in vivo by applying sequential layers of similarly or differentially doped gel or other scaffold material to the target site. For example, the device is formed by sequentially injecting the next, inner layer into the center of the previously injected material using a needle, thereby forming concentric spheroids. In certain embodiments, non-concentric compartments are formed by injecting material into different locations in a previously injected layer. A multi-headed injection device extrudes compartments in parallel and simultaneously. The layers are made of similar or different biomaterials differentially doped with pharmaceutical compositions. Alternatively, compartments self-organize based on their hydro-philic/phobic characteristics or on secondary interactions within each compartment. In certain embodiments, multicomponent scaffolds are optionally constructed in concentric layers each of which is characterized by different physical qualities such as the percentage of polymer, the percentage of crosslinking of polymer, chemical composition of the hydrogel, pore size, porosity, and pore architecture, stiffness, toughness, ductility, viscoelasticity, the growth factors, the differentiation factors, and/or homing factors incorporated therein and/or any other compositions incorporated therein.

### Hydrogel and Cryogel Scaffolds

In certain embodiments, the scaffolds of present invention comprise one or more hydrogels. A hydrogel is a polymer gel comprising a network of crosslinked polymer chains. A hydrogel is usually a composition comprising polymer chains that are hydrophilic. The network structure of hydrogels allows them to absorb significant amounts of water. Some hydrogels are highly stretchable and elastic; others are viscoelastic. Hydrogel are sometimes found as a colloidal gel in which water is the dispersion medium. In certain embodiments, hydrogels are highly absorbent (they can contain over 99% water (v/v)) natural or synthetic polymers that possess a degree of flexibility very similar to natural tissue, due to their significant water content. In certain embodiments, a hydrogel may have a property that, when an appropriate shear stress is applied, the deformable hydrogel is dramatically and reversibly compressed (up to 95% of its volume), resulting in injectable macroporous preformed scaffolds. Hydrogels have been used for therapeutic applications, e.g., as vehicles for in vivo delivery of therapeutic agents, such as small molecules, cells and biologics. Hydrogels are commonly produced from polysaccharides, such as alginates. The polysaccharides may be chemically manipulated to modulate their properties and properties of the resulting hydrogels.

The hydrogels of the present invention may be either porous or non-porous. Preferably the compositions of the invention are formed of porous hydrogels. For example, the hydrogels may be nanoporous wherein the diameter of the pores is less than about 10 nm; microporous wherein the diameter of the pores is preferably in the range of about 100 nm-20 μm; or macroporous wherein the diameter of the pores is greater than about 20 μm, more preferably greater than about 100 μm and even more preferably greater than about 400 μm. In certain embodiments, the hydrogel is macroporous with pores of about 20-80 μm in diameter. In certain embodiments, the hydrogel is macroporous with pores of about 50-80 μm in diameter. In certain embodiments, the hydrogel is macroporous with aligned pores of about 400-500 μm in diameter. Methods of preparing porous hydrogel products are known in the art. (See, e.g., U.S. Pat. No. 6,511,650, incorporated herein by reference).

The hydrogel may be constructed out of a number of different rigid, semi-rigid, flexible, gel, self-assembling, liquid crystalline, or fluid compositions such as peptide polymers, polysaccharides, synthetic polymers, hydrogel materials, ceramics (e.g., calcium phosphate or hydroxyapatite), proteins, glycoproteins, proteoglycans, metals and metal alloys. The compositions are assembled into hydrogels using methods known in the art, e.g., injection molding, lyophilization of preformed structures, printing, self-assembly, phase inversion, solvent casting, melt processing, gas foaming, fiber forming/processing, particulate leaching or a combination thereof. The assembled devices are then implanted or administered to the body of an individual to be treated.

The composition comprising a hydrogel may be assembled in vivo in several ways. The hydrogel is made from a gelling material, which is introduced into the body in its ungelled form where it gels in situ. Exemplary methods of delivering components of the composition to a site at which assembly occurs include injection through a needle or other extrusion tool, spraying, painting, or methods of deposit at a tissue site, e.g., delivery using an application device inserted through a cannula. In some embodiments, the ungelled or unformed hydrogel material is mixed with at least one pharmaceutical composition prior to introduction into the body or while it is introduced. The resultant in vivo/in situ assembled device, e.g., hydrogel, contains a mixture of the at least one pharmaceutical composition.

In situ assembly of the hydrogel may occur as a result of spontaneous association of polymers or from synergistically or chemically catalyzed polymerization. Synergistic or chemical catalysis is initiated by a number of endogenous factors or conditions at or near the assembly site, e.g., body temperature, ions or pH in the body, or by exogenous factors or conditions supplied by the operator to the assembly site, e.g., photons, heat, electrical, sound, or other radiation directed at the ungelled material after it has been introduced. The energy is directed at the hydrogel material by a radiation beam or through a heat or light conductor, such as a wire or fiber optic cable or an ultrasonic transducer. Alternatively, a shear-thinning material, is used which re-cross links after the shear force exerted upon it, for example by its passage through a needle, has been relieved.

In some embodiments, the hydrogel may be assembled ex vivo. In some embodiments, the hydrogel is injectable. For example, the hydrogels are created outside of the body as macroporous scaffolds. Upon injection into the body, the pores collapse causing the gel to become very small and allowing it to fit through a needle. See, e.g., WO2012/149358; and Bencherif et al., 2012, Proc. Natl. Acad. Sci. USA 109.48:19590-5, the content of which are incorporated herein by reference).

Suitable hydrogels for both in vivo and ex vivo assembly of hydrogel devices are well known in the art and described, e.g., in Lee et al., 2001, Chem. Rev. 7:1869-1879. The peptide amphiphile approach to self-assembly assembly is described, e.g., in Hartgerink et al., 2002, Proc. Natl. Acad. Sci. USA 99:5133-5138. A method for reversible gellation following shear thinning is exemplified in Lee et al., 2003, Adv. Mat. 15:1828-1832.

In certain embodiments, exemplary hydrogels are comprised of materials that are compatible with encapsulation of materials including polymers, nanoparticles, polypeptides, and cells. Exemplary hydrogels are fabricated from alginate, polyethylene glycol (PEG), PEG-acrylate, agarose, hyaluronic acid, or synthetic protein (e.g., collagen or engineered proteins (i.e., self-assembly peptide-based hydrogels)). For example, a commercially available hydrogel includes BD™ PuraMatrix™. BD™ PuraMatrix™ Peptide Hydrogel is a synthetic matrix that is used to create defined three dimensional (3D) micro-environments for cell culture.

In some embodiments, the hydrogel is a biocompatible polymer matrix that is biodegradable in whole or in part. Examples of materials which can form hydrogels include alginates and alginate derivatives, polylactic acid, polyglycolic acid, poly(lactic-co-glycolic acid) (PLGA) polymers, gelatin, collagen, agarose, hyaluronic acid, hyaluronic acid derivative, natural and synthetic polysaccharides, polyamino acids such as polypeptides particularly poly(lysine), polyesters such as polyhydroxybutyrate and poly-epsilon.-caprolactone, polyanhydrides; polyphosphazines, poly(vinyl alcohols), poly(alkylene oxides) particularly poly(ethylene oxides), poly(allylamines) (PAM), poly(acrylates), modified styrene polymers such as poly(4-aminomethylstyrene), pluronic polyols, polyoxamers, poly(uronic acids), poly(vinylpyrrolidone), and copolymers of the above, including graft copolymers. Synthetic polymers and naturally-occurring polymers such as, but not limited to, collagen, fibrin, hyaluronic acid, agarose, and laminin-rich gels may also be used. The term “derivative,” as used herein, refers to a compound that is derived from a similar compound by a chemical reaction. For example, oxidized alginate, which is derived from alginate through oxidization reaction, is a derivative of alginate,

The implantable composition can have virtually any regular or irregular shape including, but not limited to, spheroid, cubic, polyhedron, prism, cylinder, rod, disc, or other geometric shape. Accordingly, in some embodiments, the implant is of cylindrical form from about 0.5 to about 10 mm in diameter and from about 0.5 to about 10 cm in length. Preferably, its diameter is from about 1 to about 5 mm and its length from about 1 to about 5 cm.

In some embodiments, the compositions of the invention are of spherical form. When the composition is in a spherical form, its diameter can range, in some embodiments, from about 20 μm to about 30 μm. In an exemplary embodiment, the diameter is from about 0.5 mm to about 50 mm in diameter. In some embodiments, a spherical implant's diameter is from about 5 to about 30 mm. In an exemplary embodiment, the diameter is from about 10 to about 25 mm.

In certain embodiments, the scaffold comprises click-hydrogels and/or click-cryogels. A click hydrogel or cryogel is a gel in which cross-linking between hydrogel or cryogel polymers is facilitated by click reactions between the polymers. Each polymer may contain one of more functional groups useful in a click reaction. Given the high level of specificity of the functional group pairs in a click reaction, active compounds can be added to the preformed device prior to or contemporaneously with formation of the hydrogel device by click chemistry. Non-limiting examples of click reactions that may be used to form click-hydrogels include Copper I catalyzed azide-alkyne cycloaddition, strain-promoted assize-alkyne cycloaddition, thiol-ene photocoupling, Diels-Alder reactions, inverse electron demand Diels-Alder reactions, tetrazole-alkene photo-click reactions, oxime reactions, thiol-Michael addition, and aldehyde-hydrazide coupling. Non-limiting aspects of click hydrogels are described in Jiang et al., 2014, Biomaterials, 35:4969-4985, the entire content of which is incorporated herein by reference.

In various embodiments, a click alginate is utilized (see, e.g., PCT International Patent Application Publication No. WO 2015/154078 published Oct. 8, 2015, hereby incorporated by reference in its entirety).

In certain embodiments, a hydrogel (e.g., cryogel) system can deliver one or more agent (e.g., a growth factor such as BMP-2, and/or a differentiation factor, such as a DLL-4, while creating a space for cells (e.g., stem cells such as hematopoietic stem cells (HSC) infiltration and trafficking). In some embodiments, the hydrogel (e.g., cryogel) system according to the present invention delivers BMP-2, which acts as a hematopoietic stem cell (HSC) and/or hematopoietic progenitor cell enhancement/recruitment factor, and DLL-4 as a differentiation factor, which facilitates T cell lineage specification of hematopoietic stem cell and/or hematopoietic progenitor cells. In certain embodiments, a hydrogel (e.g., cryogel) system according to the present invention is a cell-free biomaterial-based bone marrow cryogels (BMC), fabricated from click-alginate and comprising BMP-2 to induce bone formation as well as conjugated DLL-4 protein.

In some embodiments, a cryogel composition, e.g., formed of MA-alginate, can function as a delivering platform by creating a local niche, such as a specific niche for enhancing T-lineage specification. In some embodiments, the cryogel creates a local niche in which the encounter of cells, such as recruited stem cells or progenitor cells, and various exemplary agent of the invention, such as the growth factor and/or differentiation factor can be controlled. In certain embodiments, the cells and the exemplary agents of the present invention are localized into a small volume, and the contacting of the cells and the agents can be quantitatively controlled in space and time.

In certain embodiments, the hydrogel (e.g., cryogel) can be engineered to coordinate the delivery of both growth factor and differentiation factor in space and time, potentially enhancing overall immune modulation performance by adjusting the differentiation and/or specification of recruited cells, such as hematopoietic stem cells or progenitor cells. In certain embodiments, the cells and growth factor/differentiation factor are localized into a small volume, and the delivery of factors in space and time can be quantitatively controlled. As the growth/differentiation factors are released locally, few systemic effects are anticipated, in contrast to systemically delivered agents, such as growth factors.

Examples of polymer compositions from which the cryogel or hydrogel is fabricated are described throughout the present disclosure, and include alginate, hyaluronic acid, gelatin, heparin, dextran, carob gum, PEG, PEG derivatives including PEG-co-PGA and PEG-peptide conjugates. The techniques can be applied to any biocompatible polymers, e.g., collagen, chitosan, carboxymethylcellulose, pullulan, polyvinyl alcohol (PVA), Poly(2-hydroxyethyl methacrylate) (PHEMA), Poly(N-isopropylacrylamide) (PNIPAAm), or Poly(acrylic acid) (PAAc). For example, in a particular embodiment, the composition comprises an alginate-based hydrogel/cryogel. In another example, the scaffold comprises a gelatin-based hydrogel/cryogel.

Cryogels are a class of materials with a highly porous interconnected structure that are produced using a cryotropic gelation (or cryogelation) technique. Cryogels also have a highly porous structure. Typically, active compounds are added to the cryogel device after the freeze formation of the pore/wall structure of the cryogel. Cryogels are characterized by high porosity, e.g., at least about 50, 55, 60, 65, 70, 75, 80, 85, 90, or 95% pores with thin pore walls that are characterized by high density of polymer crosslinking. As used herein, the term “porosity” refers to the percentage of the volume of pores to the volume of the scaffold. It is intended that values and ranges intermediate to the recited values are part of this invention. The walls of cryogels are typically dense and highly cross-linked, enabling them to be compressed through a needle into a subject without permanent deformation or substantial structural damage.

In various embodiments, the pore walls comprise at least about 10, 15, 20, 25, 30, 35, or 40% (w/v) polymer. It is intended that values and ranges intermediate to the recited values are part of this invention. In other embodiments, the pore walls comprise about 10-40% polymer. In some embodiments, a polymer concentration of about 0.5-4% (w/v) (before the cryogelation) is used, and the concentration increases substantially upon completion of cryogelation. Non-limiting aspects of cryogel gelation and the increase of polymer concentration after cryogelation are discussed in Beduer et al., 2015 Advanced Healthcare Materials 4.2:301-312, the entire content of which is incorporated herein by reference.

In certain embodiments, cryogelation comprises a technique in which polymerization-crosslinking reactions are conducted in quasi-frozen reaction solution. Non-limiting examples of cryogelation techniques are described in U.S. Patent Application Publication No. 20140227327, published Aug. 14, 2014, the entire content of which is incorporated herein by reference. An advantage of cryogels compared to conventional macroporous hydrogels obtained by phase separation is their high reversible deformability. Cryogels may be extremely soft but can be deformed and reform their shape. In certain embodiments, cryogels can be very tough, can withstand high levels of deformations, such as elongation and torsion and can also be squeezed under mechanical force to drain out their solvent content. The improved deformability properties of alginate cryogels originate from the high crosslinking density of the unfrozen liquid channels of the reaction system.

In the cryogelation process, during freezing of the macromonomer (e.g., methacrylated alginate) solution, the macromonomers and initiator system (e.g., APS/TEMED) are expelled from the ice concentrate within the channels between the ice crystals, so that the reactions only take place in these unfrozen liquid channels. After polymerization and, after melting of ice, a porous material is produced whose microstructure is a negative replica of the ice formed. Ice crystals act as porogens. Desired pore size is achieved, in part, by altering the temperature of the cryogelation process. For example, the cryogelation process is typically carried out by quickly freezing the solution at −20° C. Lowering the temperature to, e.g., −80° C., would result in more ice crystals and lead to smaller pores. In some embodiments, the cryogel is produced by cryo-polymerization of at least methacrylated (MA)-alginate and MA-PEG. In some embodiments, the cryogel is produced by cryo-polymerization of at least MA-alginate, the growth factor, the differentiation factor, and MA-PEG.

In some embodiments, the invention also features gelatin scaffolds, e.g., gelatin hydrogels such as gelatin cryogels, which are a cell-responsive platform for biomaterial-based therapy. Gelatin is a mixture of polypeptides that is derived from collagen by partial hydrolysis. These gelatin scaffolds have distinct advantages over other types of scaffolds and hydrogels/cryogels. For example, the gelatin scaffolds of the invention support attachment, proliferation, and survival of cells and are degraded by cells, e.g., by the action of enzymes such as matrix metalloproteinases (MMPs) (e.g., recombinant matrix metalloproteinase-2 and -9).

In certain embodiments, prefabricated gelatin cryogels rapidly reassume their approximately original shape (“shape memory”) when injected subcutaneously into a subject (e.g., a mammal such as a human, dog, cat, pig, or horse) and elicit little or no harmful host immune response (e.g., immune rejection) following injection.

In some embodiments, the hydrogel (e.g., cryogel) comprises polymers that are modified, e.g., sites on the polymer molecule are modified with a methacrylic acid group (methacrylate (MA)) or an acrylic acid group (acrylate). Exemplary modified hydrogels/cryogels are MA-alginate (methacrylated alginate) or MA-gelatin. In the case of MA-alginate or MA-gelatin, 50% corresponds to the degree of methacrylation of alginate or gelatin. This means that every other repeat unit contains a methacrylated group. The degree of methacrylation can be varied from about 1% to about 100%. Preferably, the degree of methacrylation varies from about 1% to about 90%.

In certain embodiments, polymers can also be modified with acrylated groups instead of methacrylated groups. The product would then be referred to as an acrylated-polymer. The degree of methacrylation (or acrylation) can be varied for most polymers. However, some polymers (e.g., PEG) maintain their water-solubility properties even at 100% chemical modification. After crosslinking, polymers normally reach near complete methacrylate group conversion indicating approximately 100% of cross-linking efficiency. As used herein, the term “cross-linking efficiency” refers to the percentage of macromonomers that are covalently linked. For example, the polymers in the hydrogel are 50-100% crosslinked (covalent bonds). The extent of crosslinking correlates with the durability of the hydrogel. Thus, a high level of crosslinking (90-100%) of the modified polymers is desirable.

For example, the highly crosslinked hydrogel/cryogel polymer composition is characterized by at least about 50% polymer crosslinking (e.g., about 75%, 80%, 85%, 90%, 95%, 98%, 99%, or 100%; it is intended that values and ranges intermediate to the recited values are part of this invention.). The high level of crosslinking confers mechanical robustness to the structure. Preferably, the percentage of crosslinking is less than about 100%. The composition is formed using a free radical polymerization process and a cryogelation process. For example, the cryogel is formed by cryopolymerization of methacrylated gelatin, methacrylated alginate, or methacrylated hyaluronic acid. In some embodiments, the cryogel comprises a methacrylated gelatin macro monomer or a methacrylated alginate macromonomer at concentration of about 1.5% (w/v) or less (e.g., about 1.5%, 1.4%, 1.3%, 1.2%, 1.1%, 1%, 0.9%, 0.8%, 0.7%, 0.6%, 0.5%, 0.4%, 0.3%, 0.2% or less; it is intended that values and ranges intermediate to the recited values are part of this invention.). In some embodiments, the methacrylated gelatin or alginate macromonomer concentration is about 1% (w/v).

In certain embodiments, the cryogel comprises at least about 75% (v/v) pores, e.g., about 80%, 85%, 90%, 91%, 92%, 93%, 94%, 95%, 96%, 97%, 98%, or 99% (v/v) or more pores. It is intended that values and ranges intermediate to the recited values are part of this invention. In some embodiments, the pores are interconnected. Interconnectivity is important to the function of the hydrogel and/or cryogel, as without interconnectivity, water would become trapped within the gel. Interconnectivity of the pores permits passage of water (and other compositions such as cells and compounds) in and out of the structure. In certain embodiments, in a fully hydrated state, the hydrogel (e.g., cryogel) comprises at least about 90% water (volume of water/volume of the scaffold) (e.g., between about 90-99%, at least about 92%, 95%, 97%, 99%, or more). For example, at least about 90% (e.g., at least about 92%, 95%, 97%, 99%, or more) of the volume of the cryogel is made of liquid (e.g., water) contained in the pores. It is intended that values and ranges intermediate to the recited values are part of this invention. In certain embodiments, in a compressed or dehydrated hydrogel, up to about 50%, 60%, 70% of that water is absent, e.g., the cryogel comprises less than about 25% (e.g., about 20%, 15%, 10%, 5% or less) water.

In certain embodiments, the cryogels of the invention comprise pores large enough for a cell to travel through. For example, the cryogel contains pores of about 20 to about 500 μm in diameter, e.g., about 20 to about 30 μm, about 30 to about 150 μm, about 50 to about 500 μm, about 50 to about 450 μm, about 100 to about 400 μm, about 200 to about 500 μm. In some embodiments, the hydrated pore size is about 1 to about 500 μm (e.g., about 10 to about 400 μm, about 20 to about 300 μm, about 50 to about 250 μm). In certain embodiments, the cryogel contains pores about 20 to about 80 μm in diameter. In certain embodiments, the cryogel contains pores about 50 to about 80 μm in diameter.

In some embodiments, injectable hydrogels or cryogels are further functionalized by addition of a functional group selected from the group consisting of: amino, vinyl, aldehyde, thiol, silane, carboxyl, azide, or alkyne. Alternatively or in addition, the cryogel is further functionalized by the addition of a further cross-linker agent (e.g., multiple arms polymers, salts, aldehydes, etc.). The solvent can be aqueous, and in particular, acidic or alkaline. The aqueous solvent can comprise a water-miscible solvent (e.g., methanol, ethanol, DMF, DMSO, acetone, dioxane, etc.).

For cryogels, the cryo-crosslinking may take place in a mold and the cryogels (which may be injected) can be degradable. The pore size can be controlled by the selection of the main solvent used, the incorporation of a porogen, the freezing temperature and rate applied, the crosslinking conditions (e.g. polymer concentration), and also the type and molecule weight of the polymer used. The shape of the cryogel may be dictated by a mold and can thus take on any shape desired by the fabricator, e.g., various sizes and shapes (disc, cylinders, squares, strings, etc.) are prepared by cryogenic polymerization.

Injectable cryogels can be prepared in the micrometer-scale to centimeter-scale. Exemplary volumes vary from a few hundred μm³ (e.g., about 100-500 μm³) to about 10 cm³. In certain embodiment, an exemplary scaffold composition is between about 100 μm³ to 100 mm³ in size. In various embodiments, the scaffold is between about 10 mm³ to about 100 mm³ in size. In certain embodiments, the scaffold is about 30 mm³ in size.

In some embodiments, the cryogels are hydrated, loaded with compounds and loaded into a syringe or other delivery apparatus. For example, the syringes are prefilled and refrigerated until use. In another example, the cryogel is dehydrated, e.g., lyophilized, optionally with a compound (such as a growth factor or differentiation factor) loaded in the gel and stored dry or refrigerated. Prior to administration, a cryogel-loaded syringe or apparatus may be contacted with a solution containing compounds to be delivered. For example, the barrel of the cryogel pre-loaded syringe is filled with a physiologically-compatible solution, e.g., phosphate-buffered saline (PBS). Alternatively, the cryogel may be administered to a desired anatomical site followed by administration of the physiologically-compatible solution, optionally containing other ingredients, e.g., a growth factor and/or a differentiation factor or together with one or more compounds disclosed herein. The cryogel is then rehydrated and regains its shape integrity in situ. In certain embodiments, the volume of PBS or other physiologic solution administered following cryogel placement is generally about 10 times the volume of the cryogel itself.

The cryogel also has the advantage that, upon compression, the cryogel composition maintains structural integrity and shape memory properties. For example, the cryogel is injectable through a hollow needle. For example, the cryogel returns to its approximately original geometry after traveling through a needle (e.g., a 16 gauge (G) needle, e.g., having a 1.65 mm inner diameter). Other exemplary needle sizes are 16-gauge, an 18-gauge, a 20-gauge, a 22-gauge, a 24-gauge, a 26-gauge, a 28-gauge, a 30-gauge, a 32-gauge, or a 34-gauge needle. Injectable cryogels have been designed to pass through a hollow structure, e.g., very fine needles, such as 18-30 G needles. In certain embodiments, the cryogel returns to its approximately original geometry after traveling through a needle in a short period of time, such as less than about 10 seconds, less than about 5 seconds, less than about 2 seconds, or less than about 1 second.

The cryogels may be injected to a subject using any suitable injection device. For example, the cryogels may be injected using syringe through a needle. A syringe may include a plunger, a needle, and a reservoir that comprises compositions of the present invention. The injectable cryogels may also be injected to a subject using a catheter, a cannula, or a stent.

The injectable cryogels may be molded to a desired shape, in the form of rods, square, disc, spheres, cubes, fibers, foams. In some cases, the cryogel is in the shape of a disc, cylinder, square, rectangle, or string. For example, the cryogel composition is between about 100 μm³ to 10 cm³ in size, e.g., between 10 mm³ to 100 mm³ in size. For example, the cryogel composition is between about 1 mm in diameter to about 50 mm in diameter (e.g., about 5 mm). Optionally, the thickness of the cryogel is between about 0.2 mm to about 50 mm (e.g., about 2 mm).

Three exemplary cryogel materials systems are described below.
a) Methacrylated gelatin cryogel (CryoGeIMA)—An exemplary cryogel utilized methacrylated gelatin and the results are described in detail in U.S. Patent Application Publication No. 2014-0227327, published Aug. 14, 2014, the entire contents of which are incorporated herein by reference.
b) Methacrylated alginate cryogel (CryoMAAlginate)—An exemplary cryogel utilized methacrylated alginate and the results are described in detail in U.S. Patent Application Publication No. 2014-0227327, published Aug. 14, 2014, the entire contents of which are incorporated herein by reference.
c) Click Alginate cryogel with Laponite nanoplatelets (CryoClick)—The base material is click alginate (PCT International Patent Application Publication No. WO 2015/154078 published Oct. 8, 2015, hereby incorporated by reference in its entirety). In some examples, the base material contains laponite (commercially available silicate clay used in many consumer products such as cosmetics). Laponite has a large surface area and highly negative charge density which allows it to adsorb positively charged moieties on a variety of proteins and other biologically active molecules by an electrostatic interaction, thereby allowing drug loading. When placed in an environment with a low concentration of drug, adsorbed drug releases from the laponite in a sustained manner. This system allows release of a more flexible array of various agents, e.g., growth factors, compared to the base material alone.
d) Click-gelatin hydrogels and/or click-gelatin cryogels (PCT International Patent Application Publication No. WO 2015/154078 published Oct. 8, 2015, hereby incorporated by reference in its entirety).

In various embodiments of any aspects of the invention, the scaffold comprises a click-hydrogel or click cryogel. In one embodiment, the scaffold comprises a click-alginate, a click gelatin, or a click hyaluronic acid.

Various embodiments of the present subject matter include delivery vehicles comprising a pore-forming scaffold composition. For example, pores (such as macropores) are formed in situ within a hydrogel following hydrogel injection into a subject. Pores that are formed in situ via degradation of a sacrificial porogen hydrogel within the surrounding hydrogel (bulk hydrogel) facilitate recruitment and trafficking of cells, as well as the release of any composition or agent of the present invention, for example, a growth factor, such as BMP-2, a differentiation factor, or a homing factor, or any combination thereof. In some embodiments, the sacrificial porogen hydrogel, the bulk hydrogel, or both the sacrificial porogen hydrogel and the bulk hydrogel may comprise any composition or agent of the present invention, for example, a growth factor, a differentiation factor, and/or, a homing factor, or any combination thereof.

In various embodiments, the pore-forming composition becomes macroporous over time when resident in the body of a recipient animal such as a mammalian subject. For example, the pore-forming composition may comprise a sacrificial porogen hydrogel and a bulk hydrogel, wherein the sacrificial porogen hydrogel degrades at least about 10% faster (e.g., at least about 15%, at least about 20%, at least about 25%, at least about 30%, at least about 35%, at least about 40%, at least about 45%, or at least about 50% faster) than the bulk hydrogel. It is intended that values and ranges intermediate to the recited values are part of this invention. The sacrificial porogen hydrogel may degrade leaving macropores in its place. In certain embodiments, the macropores are open interconnected macropores. In some embodiments, the sacrificial porogen hydrogel may degrade more rapidly than the bulk hydrogel, because the sacrificial porogen hydrogel (i) is more soluble in water (comprises a lower solubility index), (ii) is cross-linked to protease-mediated degradation motifs as described in U.S. Patent Application Publication No. 2005-0119762, published Jun. 2, 2005 (incorporated herein by reference in its entirety), (iii) comprises a shorter polymer that degrades more quickly compared to that of a longer bulk hydrogel polymer, (iv) is modified to render it more hydrolytically degradable than the bulk hydrogel (e.g., by oxidation), and/or (v) is more enzymatically degradable compared to the bulk hydrogel.

In various embodiments, a scaffold is loaded (e.g., soaked with) with one or more active compounds after polymerization. In certain embodiments, device or scaffold polymer forming material is mixed with one or more active compounds before polymerization. In some embodiments, a device or scaffold polymer forming material is mixed with one or more active compounds before polymerization, and then is loaded with more of the same or one or more additional active compounds after polymerization.

In some embodiments, pore size or total pore volume of a composition or scaffold is selected to influence the release of compounds from the device or scaffold. Exemplary porosities (e.g., nanoporous, microporous, and macroporous scaffolds and devices) and total pore volumes (e.g., about 5, 10, 15, 20, 25, 30, 35, 40, 45, 50, 55, 60, 65, 70, 75, 80, 85, 90, 95% or more of the volume of the scaffold) are described herein. It is intended that values and ranges intermediate to the recited values are part of this invention. Increased pore size and total pore volume increases the amount of compounds that can be delivered into or near a tissue, such as bone marrow. In some embodiments, a pore size or total pore volume is selected to increase the speed at which active ingredients exit the composition or scaffold. In various embodiments, an active ingredient may be incorporated into the scaffold material of a hydrogel or cryogel, e.g., to achieve continuous release of the active ingredient from the scaffold or device over a longer period of time compared to active ingredient that may diffuse from a pore cavity.

Porosity influences recruitment of the cells into devices and scaffolds and the release of substances from devices and scaffolds. Pores may be, e.g., nanoporous, microporous, or macroporous. For example, the diameter of nanopores is less than about 10 nm. Micropores are in the range of about 100 nm to about 20 μm in diameter. Macropores are greater than about 20 μm (e.g., greater than about 100 μm or greater than about 400 μm) in diameter. Exemplary macropore sizes include about 50 μm, about 100 μm, about 150 μm, about 200 μm, about 250 μm, about 300 μm, about 350 μm, about 400 μm, about 450 μm, about 500 μm, about 550 μm, and about 600 μm in diameter. It is intended that values and ranges intermediate to the recited values are part of this invention. Macropores are those of a size that permit a eukaryotic cell to traverse into or out of the composition. In one example, a macroporous composition has pores of about 400 μm to about 500 μm in diameter. The preferred pore size depends on the application. In certain embodiments, the pores have a diameter of about 20 μm to about 80 μm. In certain embodiments, the pores have a diameter of about 50 μm to about 80 μm.

In various embodiments, the composition is manufactured in one stage in which one layer or compartment is made and infused or coated with one or more compounds. Exemplary bioactive compositions comprise polypeptides or polynucleotides. In certain embodiments, the composition is manufactured in two or more (3, 4, 5, 6, . . . 10 or more) stages in which one layer or compartment is made and infused or coated with one or more compounds followed by the construction of second, third, fourth or more layers, which are in turn infused or coated with one or more compounds in sequence. In some embodiments, each layer or compartment is identical to the others or distinguished from one another by the number or mixture of bioactive compositions as well as distinct chemical, physical and biological properties. Polymers may be formulated for specific applications by controlling the molecular weight, rate of degradation, and method of scaffold formation. Coupling reactions can be used to covalently attach bioactive agent, such as the differentiation factor to the polymer backbone.

In some embodiments, one or more compounds is added to the scaffold compositions using a known method including surface absorption, physical immobilization, e.g., using a phase change to entrap the substance in the scaffold material. For example, a growth factor is mixed with the scaffold composition while it is in an aqueous or liquid phase, and after a change in environmental conditions (e.g., pH, temperature, ion concentration), the liquid gels or solidifies thereby entrapping the bioactive substance. In some embodiments, covalent coupling, e.g., using alkylating or acylating agents, is used to provide a stable, long term presentation of a compound on the scaffold in a defined conformation. Exemplary reagents for covalent coupling of such substances are provided in the table below.

TABLE 1
Methods to Covalently Couple Peptides/Proteins to Polymers

| Functional | | Reacting |
|---|---|---|
| Group of | Coupling Reagents and | Groups on |
| Polymer | Cross-Liner | Proteins/Peptides |
| —OH | Cyanogen bromide (CNBr) | —NH₂ |
| | Cyanuric chloride | |
| | 4-( 4,6-Dimethoxy-1,3,5- | |
| | triazin-2-yl)-4- | |
| | methylmorpholinium chloride | |
| | (DMT-MM) | |
| —NH₂ | Diisocyanate compounds | —NH₂ |
| | Diisothoncyanate | —OH |
| | compounds | |
| | Glutaraldehyde Succinic | |
| | anhydride | |
| —NH₂ | Nitrous Acid | —NH₂ |
| | Hydrazine + nitrous acid | —SH |
| | | —Ph—OH |
| —NH₂ | Carbodiimide compounds | —COOH |
| | (e.g., EDC, DCC)[a] | |
| | DMT-MM | |
| —COOH | Thiony I chloride | —NH₂ |
| | N-hydroxysuccinimide | |
| | N-hydroxysulfosuccinimide + | |
| | EDC | |
| —SH | Disulfide compound | —SH |

[a]EDC: 1-ethyl-3-(3-dimethylaminopropyl) carbodiimide hydrochloride; DCC: dicyclohexylcarbodiimide

In some embodiments, one or more compounds is added to the scaffold compositions using a known method including click chemistry. In some embodiments, one or more compounds may be covalently linked to the scaffold utilizing click chemistry. The methods of covalently binding or coupling include, but are not limited to, avidin-biotin reaction, azide and dibenzocycloocytne chemistry, tetrazine and transcyclooctene chemistry, tetrazine and norbornene chemistry, or di-sulfide bond.

### Alginate Scaffolds

In certain embodiments, the composition of the invention comprises an alginate hydrogel. Alginates are versatile polysaccharide based polymers that may be formulated for specific applications by controlling the molecular weight, rate of degradation and method of scaffold formation. Alginate polymers are comprised of two different monomeric units, (1-4)-linked β-D-mannuronic acid (M units) and α L-guluronic acid (G units) monomers, which can vary in proportion and sequential distribution along the polymer chain. Alginate polymers are polyelectrolyte systems which have a strong affinity for divalent cations (e.g., Ca⁺², Mg⁺², Ba⁺²) and form stable hydrogels when exposed to these molecules. See Martinsen A., et al., 1989, Biotech. & Bioeng., 33:79-89). For example, calcium cross-linked alginate hydrogels are useful for dental applications, wound dressings chondrocyte transplantation and as a matrix for other cell types. Without wishing to be bound by theory, it is believed that G units are preferentially crosslinked using calcium crosslinking, whereas click reaction based crosslinking is more indiscriminate with respect to G units or M units (i.e., both G and M units can be crosslinked by click chemistry). Alginate scaffolds and the methods for making them are known in the art. Sec, e.g., International Patent Application Publication No. WO2017/075055 A1, published on May 4, 2017, the entire contents of which are incorporated herein by reference.

The alginate polymers useful in the context of the present invention can have an average molecular weight from about 20 kDa to about 500 kDa, e.g., from about 20 kDa to about 40 kDa, from about 30 kDa to about 70 kDa, from about 50 kDa to about 150 kDa, from about 130 kDa to about 300 kDa, from about 230 kDa to about 400 kDa, from about 300 kDa to about 450 kDa, or from about 320 kDa to about 500 kDa. In one example, the alginate polymers useful in the present invention may have an average molecular weight of about 32 kDa. In another example, the alginate polymers useful in the present invention may have an average molecular weight of about 265 kDa. In some embodiments, the alginate polymer has a molecular weight of less than about 1000 kDa, e.g., less than about 900 KDa, less than about 800 kDa, less than about 700 kDa, less than about 600 kDa, less than about 500 kDa, less than about 400 kDa, less than about 300 kDa, less than about 200 kDa, less than about 100 kDa, less than about 50 kDa, less than about 40 kDa, less than about 30 kDa or less than about 25 kDa. In some embodiments, the alginate polymer has a molecular weight of about 1000 kDa, e.g., about 900 kDa, about 800 kDa, about 700 kDa, about 600 kDa, about 500 kDa, about 400 kDa, about 300 kDa, about 200 kDa, about 100 kDa, about 50 kDa, about 40 kDa, about 30 kDa or about 25 kDa. In one embodiment, the molecular weight of the alginate polymers is about 20 kDa.

Coupling reactions can be used to covalently attach bioactive agent, such as an atom, a chemical group, a nucleoside, a nucleotide, a nucleobase, a sugar, a nucleic acid, an amino acid, a peptide, a polypeptide, a protein, or a protein complex, to the polymer backbone.

The term “alginate”, used interchangeably with the term “alginate polymers”, includes unmodified alginate or modified alginate. Modified alginate includes, but not limited to, oxidized alginate (e.g., comprising one or more algoxalate monomer units) and/or reduced alginate (e.g., comprising one or more algoxinol monomer units). In some embodiments, oxidized alginate comprises alginate comprising one or more aldehyde groups, or alginate comprising one or more carboxylate groups. In other embodiments, oxidized alginate comprises highly oxidized alginate, e.g., comprising one or more algoxalate units. Oxidized alginate may also comprise a relatively small number of aldehyde groups (e.g., less than 15%, e.g., 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, or 0.1% aldehyde groups or oxidation on a molar basis). It is intended that values and ranges intermediate to the recited values are part of this invention.

The term “alginate” or “alginate polymers” may also include alginate, e.g., unmodified alginate, oxidized alginate or reduced alginate, or methacrylated alginate or acrylated alginate. Alginate may also refer to any number of derivatives of alginic acid (e.g., calcium, sodium or potassium salts, or propylene glycol alginate). Sec, e.g., WO1998012228A1, hereby incorporated by reference.

### Hyaluronic Acid

In certain embodiments, the composition of the present invention comprises a hyaluronic acid hydrogel. Hyaluronic acid (HA; conjugate base hyaluronate), is an anionic, nonsulfated glycosaminoglycan distributed widely throughout connective, epithelial, and neural tissues. One of the chief components of the extracellular matrix, hyaluronic acid contributes significantly to cell proliferation and migration. Natural hyaluronic acid is an important component of articular cartilage, muscular connective tissues, and skin.

Hyaluronic acid is a polymer of disaccharides, composed of D-glucuronic acid and N-acetyl-D-glucosamine, linked via alternating β-(1→4) and β-(1→3) glycosidic bonds. Hyaluronic acid can be 25,000 disaccharide repeats in length. Polymers of hyaluronic acid can range in size from 5,000 to 20,000,000 Da. Hyaluronic acid can also contain silicon.

Hyaluronic acid is energetically stable, in part because of the stereochemistry of its component disaccharides. Bulky groups on each sugar molecule are in sterically favored positions, whereas the smaller hydrogens assume the less-favorable axial positions.

Hyaluronic acid can be degraded by a family of enzymes called hyaluronidases, which are present in many mammals, e.g., a human. Hyaluronic acid can also be degraded via non-enzymatic reactions. These include acidic and alkaline hydrolysis, ultrasonic disintegration, thermal decomposition, and degradation by oxidants.

Due to its high biocompatibility and its common presence in the extracellular matrix of tissues, hyaluronic acid is used to form hydrogels, e.g., cryogels, as a biomaterial scaffold in tissue engineering research. Hyaluronic acid hydrogels are formed through crosslinking. Hyaluronic acid can form a hydrogel, e.g., cryogel, into a desired shape to deliver therapeutic molecules into a host. Hyaluronic acids, for use in the present compositions, can be crosslinked by attaching thiols, methacrylates, hexadecylamides, and tyramines. Hyaluronic acids can also be crosslinked directly with formaldehyde or with divinylsulfone.

The term “hyaluronic acid,” includes unmodified hyaluronic acid or modified hyaluronic acid. Modified hyaluronic acid includes, but is not limited to, oxidized hyaluronic acid and/or reduced hyaluronic acid. The term “hyaluronic acid” or “hyaluronic acid polymers” may also include hyaluronic acid, e.g., unmodified hyaluronic acid, oxidized hyaluronic acid or reduced hyaluronic acid, or methacrylated hyaluronic acid or acrylated hyaluronic acid. Hyaluronic acid may also refer to any number of derivatives of hyaluronic acid.

### Porous and Pore-Forming Scaffolds

The scaffolds of the present invention may be nonporous or porous. In certain embodiments, the scaffolds of the present invention are porous. Porosity of the scaffold composition influences migration of the cells through the device. Pores may be nanoporous, microporous, or macroporous. For example, the diameter of nanopores is less than about 10 nm. Micropores are in the range of about 100 nm to about 20 μm in diameter. Macropores are greater than about 20 μm (e.g., greater than about 100 μm or greater than about 400 μm) in diameter. Exemplary macropore sizes include about 50 μm, 100 μm, 150 μm, 200 μm, 250 μm, 300 μm, 350 μm, 400 μm, 450 μm, 500 μm, 550 μm, and 600 μm in diameter. It is intended that values and ranges intermediate to the recited values are part of this invention. Macropores are of a size that permits a eukaryotic cell to traverse into or out of the composition. In certain embodiments, a macroporous composition has pores of about 400 μm to 500 μm in diameter. The size of pores may be adjusted for different purpose. For example, for cell recruitment and cell release, the pore diameter may be greater than 50 μm. In certain embodiments, a macroporous composition has pores of about 20 μm-about 80 μm in diameter. In certain embodiments, a macroporous composition has pores of about 50 μm-about 80 μm in diameter.

In some embodiments, the scaffolds contain pores before the administration into a subject. In some embodiments, the scaffolds comprise a pore-forming scaffold composition. Pore-forming scaffolds and the methods for making pore-forming scaffolds are known in the art. Sec, e.g., U.S. Patent Publication US2014/0079752A1, the content of which is incorporated herein by reference. In certain embodiments, the pore-forming scaffolds are not initially porous, but become macroporous over time resident in the body of a recipient animal such as a mammalian subject. In certain embodiments, the pore-forming scaffolds are hydrogel scaffolds. The pore may be formed at different time, e.g., after about 12 hours, or 1, 3, 5, 7, or 10 days or more after administration, i.e., resident in the body of the subject.

In certain embodiments, the pore-forming scaffolds comprise a first hydrogel and a second hydrogel, wherein the first hydrogel degrades at least about 10% faster (e.g., at least about 15%, at least about 20%, at least about 25%, at least about 30%, at least about 35%, at least about 40%, at least about 45%, at least about 50% faster, at least about 2 times faster, or at least about 5 times faster) than the second hydrogel. It is intended that values and ranges intermediate to the recited values are part of this invention. In certain embodiments, the first hydrogel comprises a porogen that degrades leaving a pore in its place. For example, the first hydrogel is a porogen and the resulting pore after degradation in situ is within 25% of the size of the initial porogen, e.g., within 20%, within 15%, or within 10% of the size of the initial porogen. Preferably, the resulting pore is within 5% of the size of the initial porogen. It is intended that values and ranges intermediate to the recited values are part of this invention. The first hydrogel may degrade faster than the second hydrogel due to the difference in their physical, chemical, and/or biological properties. In certain embodiments, the first hydrogel degrades more rapidly than the second hydrogel, because the first hydrogel is more soluble in water (comprises a lower solubility index). In certain embodiments, the first hydrogel degrades more rapidly because it is cross-linked to protease-mediated degradation motifs as described in U.S. Patent Publication US2005/0119762A1, the content of which is incorporated herein by reference.

In certain embodiments, the molecular mass of the polymers used to form the first hydrogel composition (a porogen) is approximately 50 kilodaltons (kDa), and the molecular mass of the polymers used to form the second hydrogel composition (bulk) is approximately 250 kDa. A shorter polymer (e.g., that of a porogen) degrades more quickly compared to that of a longer polymer (e.g., that of the bulk composition). In certain embodiments, a composition is modified to render it more hydrolytically degradable by virtue of the presence of sugar groups (e.g., approximately 3-10% sugar of an alginate composition). In certain embodiments, the porogen hydrogel is chemically modified, such as oxidized, to render it more susceptible to degradation. In some embodiments, the porogen hydrogel is more enzymatically degradable compared to the bulk hydrogel. The composite (first and second hydrogel) composition is permeable to bodily fluids, e.g., containing an enzyme which is exposed to the composition and degrades the porogen hydrogel. In some embodiments, the second hydrogel is cross-linked around the first hydrogel, i.e., the porogens (first hydrogel) are completely physically entrapped in the bulk (second) hydrogel.

The click reagents disclosed herein can be provided in the bulk hydrogel or the porogen hydrogel. In exemplary embodiments, the click reagents, e.g., polymers or nanoparticles, are provided in the bulk hydrogel.

In certain embodiments, hydrogel micro-beads (“porogens”) are formed. Porogens are encapsulated into a “bulk” hydrogel that is either non-degradable or which degrades at a slower rate compared to the porogens. Immediately after hydrogel formation, or injection into the desired site in vivo, the composite material lacks pores. Subsequently, porogen degradation causes pores to form in situ. The size and distribution of pores are controlled during porogen formation, and mixing with the polymers which form the bulk hydrogel.

In some embodiments, the polymer utilized in the pore-forming scaffolds is naturally-occurring or synthetically made. In one example, both the porogens and bulk hydrogels are formed from alginate.

In certain embodiments, the alginate polymers suitable for porogen formation have a molecular weight from 5,000 to 500,000 Daltons. The polymers are optionally further modified (e.g., by oxidation with sodium periodate, (Bouhadir et al., 2001, Biotech. Prog. 17:945-950, hereby incorporated by reference), to facilitate rapid degradation. In certain embodiments, the polymers are crosslinked by extrusion through a nebulizer with co-axial airflow into a bath of divalent cation (for example, Ca²⁺ or Ba²⁺) to form hydrogel micro-beads. Higher airflow rate leads to lower the porogen diameter.

In some embodiments, the porogen hydrogel microbeads contain oxidized alginate. For example, the porogen hydrogel can contain about 1 to about 50% (w/v) oxidized alginate. In exemplary embodiments, the porogen hydrogel can contain about 1-10% oxidized alginate. In one embodiment, the porogen hydrogel contains about 7.5% oxidized alginate.

In certain embodiments, the concentration of divalent ions used to form porogens may vary from about 5 to about 500 mM, and the concentration of polymer from about 1% to about 5% by weight/volume. However, any method which produces porogens that are significantly smaller than the bulk phase is suitable. Porogen chemistry can further be manipulated to produce porogens that interact with host proteins and/or cells, or inhibit interactions with host proteins and/or cells.

The alginate polymers suitable for formation of the bulk hydrogel have a molecular weight from about 5,000 to about 500,000 Da. The polymers may be further modified (for example, by oxidation with sodium periodate), to facilitate degradation, as long as the bulk hydrogel degrades more slowly than the porogen. The polymers may also be modified to present biological cues to control cell responses (e.g., integrin binding adhesion peptides such as RGD). Either the porogens or the bulk hydrogel may also encapsulate bioactive factors such as oligonucleotides, growth factors or drugs to further control cell responses. The concentration of divalent ions used to form the bulk hydrogel may vary from about 5 to about 500 mM, and the concentration of polymer from about 1% to about 5% by weight/volume. The elastic modulus of the bulk polymer is tailored for its purpose, e.g., to recruit stem cells or progenitor cells.

Methods relevant to generating the hydrogels described herein include the following. Bouhadir et al., 1999, Polymer, 40:3575-84 (incorporated herein by reference in its entirety) describes the oxidation of alginate with sodium periodate, and characterizes the reaction. Bouhadir et al., 2001, Biotechnol. Prog., 17:945-50 (incorporated herein by reference in its entirety) describes oxidation of high molecular weight alginate to form alginate dialdehyde (alginate dialdehyde is high molecular weight (M_{w}) alginate in which a certain percent, e.g., 5%, of sugars in alginate are oxidized to form aldehydes), and application to make hydrogels degrade rapidly. Kong et al., 2002, Polymer, 43:6239-46 (incorporated herein by reference in its entirety) describes the use of gamma-irradiation to reduce the weight-averaged molecular weight (M_{w}) of guluronic acid (GA) rich alginates without substantially reducing GA content (e.g., the gamma irradiation selectively attacks mannuronic acid, MA blocks of alginate). Alginate is comprised of GA blocks and MA blocks, and it is the GA blocks that give alginate its rigidity (clastic modulus). Kong et al., 2002, Polymer, 43:6239-46 (incorporated herein by reference in its entirety) shows that binary combinations of high M_{w}, GA rich alginate with irradiated, low M_{w}, high GA alginate crosslinks with calcium to form rigid hydrogels, but which degrade more rapidly and also have lower solution viscosity than hydrogels made from the same overall weight concentration of only high M_{w}, GA rich alginate. Alsberg et al., 2003, J Dent Res, 82 (11): 903-8 (incorporated herein by reference in its entirety) describes degradation profiles of hydrogels made from irradiated, low M_{w}, GA-rich alginate, with application in bone tissue engineering. Kong et al., 2004, Adv. Mater, 16 (21): 1917-21 (incorporated herein by reference) describes control of hydrogel degradation profile by combining gamma irradiation procedure with oxidation reaction, and application to cartilage engineering.

Techniques to control degradation of hydrogen biomaterials are well known in the art. For example, Lutolf M P et al., 2003, Nat Biotechnol., 21:513-8 (incorporated herein by reference in its entirety) describes poly(ethylene glycol) based materials engineered to degrade via mammalian enzymes (MMPs). Bryant S J et al., 2007, Biomaterials, 28 (19): 2978-86 (U.S. Pat. No. 7,192,693 B2; incorporated herein by reference in its entirety) describes a method to produce hydrogels with macro-scale pores. A pore template (e.g., poly-methylmethacrylate beads) is encapsulated within a bulk hydrogel, and then acetone and methanol are used to extract the porogen while leaving the bulk hydrogel intact. Silva et al., 2008, Proc. Natl. Acad. Sci USA, 105 (38): 14347-52 (incorporated herein by reference in its entirety; US 2008/0044900) describes deployment of endothelial progenitor cells from alginate sponges. The sponges are made by forming alginate hydrogels and then freeze-drying them (ice crystals form the pores). Ali et al., 2009, Nat Mater (incorporated herein by reference in its entirety) describes the use of porous scaffolds to recruit dendritic cells and program them to elicit anti-tumor responses. Huebsch et al., 2010, Nat Mater, 9:518-26 (incorporated herein by reference in its entirety) describes the use of hydrogel elastic modulus to control the differentiation of encapsulated mesenchymal stem cells.

In some embodiments, the scaffold composition comprises open interconnected macropores. Alternatively or in addition, the scaffold composition comprises a pore-forming scaffold composition. In certain embodiments, the pore-forming scaffold composition may comprise a sacrificial porogen hydrogel and a bulk hydrogel, wherein the pore-forming scaffold composition lacks macropores. For example, the sacrificial porogen hydrogel may degrade at least 10% faster than the bulk hydrogel leaving macropores in its place following administration of said pore-forming scaffold into a subject. In some embodiments, the sacrificial porogen hydrogel is in the form of porogens that degrade to form said macropores. For example, the macropores may comprise pores having a diameter of, e.g., about 10-400 μm.

### Growth Factors

The compositions of the present invention can comprise a growth factor. The term “growth factor,” as used herein, refers to an agent that is capable of stimulating cellular growth, proliferation, healing, and/or cellular differentiation. In certain embodiments, growth factors are polypeptides. Growth factor polypeptides typically act as signaling molecules. In certain embodiments, the growth factor polypeptides are cytokines.

In certain embodiments, the growth factor can recruit a cell to the scaffold following the administration of the composition to a subject. The recruited cell may be autologous. For example, the recruited cell may be a stromal cell from the subject. In certain embodiments, the autologous cell may be a stem cell (e.g., umbilical cord stem cells) of the subject. The recruited cell may also be syngeneic, allogeneic or xenogeneic. As used herein, the term “syngeneic” refers to genetically identical, or sufficiently identical and immunologically compatible as to allow for transplantation. For example, syngeneic cells may include transplanted cells obtained from an identical twin. As used herein, the term “allogeneic” refers to cells that are genetically dissimilar, although from individuals of the same species. As used herein, the term “xenogeneic” refers to cells derived from a different species and therefore genetically different.

For example, the recruited cell may be a donor cell in a transplantation. In certain embodiments, the transplantation is a hematopoietic stem cell transplantation (HSCT). As used herein, HSCT refers to the transplantation of multipotent hematopoietic stem cells or hematopoietic progenitor cells, usually derived from bone marrow, peripheral blood, or umbilical cord blood.-HSCT may be autologous (the patient's own stem cells or progenitor cells are used), allogeneic (the stem cells or progenitor cells come from a donor), syngeneic (from an identical twin) or xenogenic (from different species).

The growth factors of the present invention may induce the formation of a tissue or organ within or around the administered composition. In certain embodiments, the tissue or organ is a bony tissue or hematopoietic tissue. The tissue formation may be restricted to the scaffold of the composition.

Methods of incorporating polypeptides (e.g., growth factor polypeptides) are known in the art. See, U.S. Pat. Nos. 8,728,456; 8,067,237; and 10,045,947; US Patent Publication No.: US20140079752; International Patent Publication No.: WO2017/136837; incorporated herein by reference in their entirety. The release of the growth factor polypeptides may be controlled. The methods of controlled release of polypeptides (e.g., growth factor polypeptides) are known in the art. See, U.S. Pat. Nos. 8,728,456; 8,067,237; 10,045,946, incorporated by reference in their entirety. In certain embodiments, the growth factors (e.g., BMP-2) may be released over an extended period of time, such as 7-30 days or longer. The controlled release of the growth factors may affect the timing of the formation of the tissue or organ within the scaffold. In certain examples, the release of the growth factors is controlled with the goal of creating a functional, active bone nodule or tissue within one to two weeks after subcutaneous injection of the compositions of the present invention.

In certain embodiments, the growth factors retain their bioactivity over an extended period of time. The term “bioactivity,” as used herein, refers to the beneficial or adverse effects of an agent, such as a growth factor. The bioactivity of the growth factor may be measured by any appropriate means. For example, the bioactivity of BMP-2 may be measured by its capacity to induce the formation of bone nodule or tissue and/or recruit cells into the scaffold. In certain example, the growth factors retain their bioactivity for at least 10 days, 12 days, 14 days, 20 days, or 30 days after the incorporation of the growth factors into the scaffold.

Exemplary growth factors include, but are not limited to, bone morphogenetic proteins (BMP), epidermal growth factor (EGF), transforming growth factor beta (TGF-β), granulocyte-colony stimulating factor (G-CSF), granulocyte-macrophage colony stimulating factor (GM-CSF), nerve growth factor (NGF), neurotrophins, Platelet-derived growth factor (PDGF), erythropoietin (EPO), thrombopoietin (TPO), myostatin (GDF-8), growth differentiation factor-9 (GDF9), acidic fibroblast growth factor (aFGF or FGF-1), basic fibroblast growth factor (bFGF or FGF-2), epidermal growth factor (EGF), hepatocyte growth factor (HGF), insulin-like growth factor (IGF), and interleukins.

In some embodiments, the growth factor comprises a protein belonging to the transforming growth factor beta (TGF-β) superfamily. As used herein, TGF-β superfamily is a large group of structurally related cell regulatory proteins. TGF-β superfamily includes four major subfamilies: the TGF-β subfamily, the bone morphogenetic proteins and the growth differentiation factors, the activing and inhibin subfamilies, and a group encompassing various divergent members. Proteins from the TGF-superfamily are active as homo- or heterodimer, the two chains being linked by a single disulfide bond. TGF-β superfamily proteins interact with a conserved family of cell surface serine/threonine-specific protein kinase receptors, and generate intracellular signals using a conserved family of proteins called SMADs. TGF-β superfamily proteins play important roles in the regulation of basic biological processes such as growth, development, tissue homeostasis and regulation of the immune system.

Exemplary TGF-β superfamily proteins include, but are not limited to, AMH, ARTN, BMP10, BMP15, BMP2, BMP3, BMP4, BMP5, BMP6, BMP7, BMP8A, BMP8B, GDF1, GDF10, GDF11, GDF15, GDF2, GDF3, GDF3A, GDF5, GDF6, GDF7, GDF8, GDF9, GDNF, INHA, INHBA, INHBB, INHBC, INHBE, LEFTY1, LEFTY2, MSTN, NODAL, NRTN, PSPN, TGF-1, TGF-β2, TGF-β3, and TGF-β4. In a particular embodiment, the growth factor is BMP2.

In certain embodiments, the growth factor comprises a bone morphogenetic protein (BMP). As used herein, a BMP is a protein belonging to a group of growth factors also known as cytokines and as metabologens. BMPs can induce the formation of bone and cartilage and constitute a group of important morphogenetic signals, orchestrating tissue architecture throughout the body. Absence or deficiency of BMP signaling may be an important factor in diseases or disorders.

In certain embodiments, the BMP is selected from a group consisting of a BMP-2, a BMP-4, a BMP-6, a BMP-7, a BMP-12, a BMP-14, and any combination thereof. In certain embodiments, the BMP is BMP-2. BMP-2 plays an important role in the development of bone and cartilage. BMP-2 can potently induce osteoblast differentiation in a variety of cell types.

In certain embodiments, the growth factor comprises a TGF-β subfamily protein. As used herein, TGF-β subfamily protein or TGF-β is a multifunctional cytokine that includes four different isoforms (TGF-β1, TGF-β2, TGF-β3, and TGF-β4). Activated TGF-complexes with other factors to form a serine/threonine kinase complex that binds to TGF-β receptors, which is composed of both type 1 and type 2 receptor subunits. After the binding of TGF-β, the type 2 receptor kinase phosphorylates and activates the type 1 receptor kinase that activates a signaling cascade. This leads to the activation of different downstream substrates and regulatory proteins, inducing transcription of different target genes that function in differentiation, chemotaxis, proliferation, and activation of many immune cells.

In certain embodiments, the growth factor comprises a TGF-β1. TGF-β1 plays a role in the induction from CD4+ T cells of both induced Tregs (iTregs), which have a regulatory function, and Tₕ17 cells, which secrete pro-inflammatory cytokines. TGF-β1 alone precipitates the expression of Foxp3 and Treg differentiation from activated T helper cells.

The growth factors, (e.g., BMP-2 or TGF-β1), may be isolated from endogenous sources or synthesized in vivo or in vitro. Endogenous growth factor polypeptides may be isolated from healthy human tissue. Synthetic growth factor polypeptides are synthesized in vivo following transfection or transformation of template DNA into a host organism or cell, e.g., a mammalian or human cell line. Alternatively, synthetic growth factor polypeptides are synthesized in vitro by cell free translation or other art-recognized methods Sambrook, J., Fritsch, E. F., and Maniatis, T., Molecular Cloning: A Laboratory Manual. Cold Spring Harbor Laboratory Press, NY, Vol. 1, 2, 3 (1989), herein incorporated by reference.

In certain embodiments, growth factor (e.g., BMP-2 or TGF-β1) polypeptides may be recombinant. In some embodiments, growth factor polypeptides are humanized derivatives of mammalian growth factor polypeptides. Exemplary mammalian species from which growth factor polypeptides are derived include, but are not limited to, mouse, rat, hamster, guinea pig, ferret, cat, dog, monkey, or primate. In some embodiments, the growth factor is a recombinant human protein. In some embodiments, the growth factor is a recombinant murine (mouse) protein. In some embodiments, the growth factor is a humanized derivative of a recombinant mouse protein.

In certain embodiments, the growth factor polypeptides may be modified to increase protein stability in vivo. In certain embodiments, the growth factor polypeptides may be engineered to be more or less immunogenic. The terms “immunogenic” and “immunogenicity” refer to the ability of a particular substance, such as a protein, an antigen, or an epitope, to provoke an immune response in the body of a human and other animal.

In certain embodiments, the growth factors may be present at between about 0.001 nmol and about 1000 nmol per scaffold, or about 0.001 and about 100 nmol per scaffold, or about 0.001 nmol and about 1 nmol per scaffold.

In some embodiments, the growth factors may be present at between about 1 ng to about 1000 micrograms per scaffold. For example, the growth factors may be present at an amount between about 1 μg and about 1000 μg, between about 1 μg and about 500 μg, between about 1 μg and about 200 μg, between about 1 μg and about 100 μg, between about 1 μg and about 50 μg, or between about 1 μg and about 10 μg.

In certain embodiments, the composition of the present invention comprises nanogram quantities of growth factors (e.g., about 1 ng to about 100 mg of BMP-2). For example, the growth factors may be present at an amount between about 5 ng and about 500 ng, between about 5 ng and about 250 ng, between about 5 ng and about 200 ng, between about 10 ng and about 200 ng, between about 25 ng and about 200 ng, between about 50 ng and 200 ng, between about 100 ng and 200 ng, and about 200 ng. Nanogram quantities of the growth factor are also released in a controlled manner. The nanogram quantities of the growth factors and/or the controlled release can contribute to reduced toxicity of the compositions and methods of the present invention as compared to other delivery system, which uses high dose of growth factors and has suboptimal release kinetics.

In various embodiments, the amount of growth factors present in a scaffold may vary according to the size of the scaffold. For example, the growth factor may be present at about 0.03 ng/mm³ (the ratio of the amount of growth factors in weight to the volume of the scaffold) to about 350 ng/mm³, such as between about 0.1 ng/mm³ and about 300 ng/mm³, between about 0.5 ng/mm³ and about 250 ng/mm³, between about 1 ng/mm³ and about 200 ng/mm³, between about 2 ng/mm³ and about 150 ng/mm³, between about 3 ng/mm³ and about 100 ng/mm³, between about 4 ng/mm³ and about 50 ng/mm³, between about 5 ng/mm³ and 25 ng/mm³, between about 6 ng/mm³ and about 10 ng/mm³, or between about 6.5 ng/mm³ and about 7.0 ng/mm³.

In some embodiments, the amount of growth factors may be present at between about 300 ng/mm³ and about 350 μg/mm³, such as between about 400 ng/mm³ and between about 300 μg/mm³, between about 500 ng/mm³ and about 200 μg/mm³, between about 1 μg/mm³ and about 100 μg/mm³, between about 5 μg/mm³ and about 50 μg/mm³, between about 10 μg/mm³ and about 25 μg/mm³.

### Differentiation Factors

The composition of the present invention can comprise a differentiation factor. As used herein, a differentiation factor is an agent that can induce the differentiation of a cell, for example, a recruited cell. In certain embodiments, the differentiation factor is a polypeptide. As used herein, “differentiation,” “cell differentiation,” “cellular differentiation,” or other similar terms refer to the process where a cell changes from one cell type to another. In certain embodiments, the cell changes to a more specialized type, e.g., from a stem cell or a progenitor cell to a T cell progenitor cell. Differentiation occurs numerous times during the development of a multicellular organism as it changes from a simple zygote to a complex system of tissues and cell types. Differentiation continues in adulthood as adult stem cells divide and create fully differentiated daughter cells during tissue repair and during normal cell turnover. Differentiation may change a cell's size, shape, membrane potential, metabolic activity, and responsiveness to signals. These changes may be due to highly controlled modifications in gene expression.

Among dividing cells, there are multiple levels of cell potency, the cell's ability to differentiate into other cell types. A greater potency indicates a larger number of cell types that can be derived. A cell that can differentiate into all cell types, including the placental tissue, is known as totipotent. A cell that can differentiate into all cell types of the adult organism is known as pluripotent. In mammals, e.g., human being, a pluripotent cell may include embryonic stem cells and adult pluripotent cells. Induced pluripotent stem (iPS) cells may be created from fibroblasts by induced expression of certain transcription factors, e.g., Oct4, Sox2, c-Myc, and KIF4. A multipotent cell is one that can differentiate into multiple different, but closely related cell types. Oligopotent cells are more restricted than multipotent, but can still differentiate into a few closely related cell types. Finally, unipotent cells can differentiate into only one cell type, but are capable of self-renewal.

In certain embodiments, the differentiation factors of the present invention induce the differentiation of stem cells or progenitor cells into T-cell progenitor cells. As used herein, the term “T cell progenitor cell” refers to a progenitor cell that ultimately can differentiate to a T lymphocyte (T cell). The term “lymphocyte,” as used herein, refers to one of the subtypes of white blood cell in a vertebrate's (e.g., human being) immune system. Lymphocytes include natural killer cells, T cells, and B cells. Lymphocytes originate from a common lymphoid progenitor during hematopoiesis, a process during which stem cells differentiate into several kinds of blood cells within the bone marrow, before differentiating into their distinct lymphocyte types.

In some embodiments, the T cell progenitor cell comprises a common lymphoid progenitor cell. The term “common lymphoid progenitor cell,” as used herein, refers to the earliest lymphoid progenitor cells, which give rise to lymphocytes including T-lineage cells, B-lineage cells, and natural killer (NK) cells. In various embodiment, the T cell progenitor cell comprises a T cell competent common lymphoid progenitor cell. The term “T cell competent common lymphoid progenitor cell,” as used herein, refers to a common lymphoid progenitor cell that differentiates into T-lineage progenitor cell. A T cell competent common lymphoid progenitor is usually characterized by lacking of biomarker Ly6D. The composition of the present invention can create an ectopic niche that mimics important features of bone marrow and induces the differentiation of stem cells or progenitor cells into T cell progenitor cells.

In certain embodiments, the lymphocytes comprise T cells. In some embodiments, the T cells are naïve T cells. As used herein, a naïve T cell is a T cell that has differentiated in bone marrow. Naïve T cells may include CD4⁺ T cells, CD8⁺ T cells, and regulatory T cells (T_{reg}).

In certain embodiments, the differentiation factors induce the differentiation of the recruited cells into T cell progenitor cells. In certain embodiments, the differentiation factors induce the differentiation of the recruited cells into T cell progenitor cells through the Notch signaling pathway. The Notch signaling pathway is a highly conserved cell signaling system present in many multicellular organisms. Mammals possess four different Notch receptors, referred to as Notch1, Notch2, Notch3, and Notch4. Notch signaling plays an important role in T cell lineage differentiation from common lymphoid progenitor cells. In certain embodiments, the differentiation factors bind to one or more Notch receptors and activates the Notch signaling pathway. In certain embodiments, the differentiation factor is selected from a group consisting of a Delta-like 1 (DLL-1), a Delta-like 2 (DLL-2), a Delta-like 3 (DLL-3), a Delta-like 3 (DLL-3), a Delta-like 4 (DLL-4), a Jagged 1, a Jagged 2, and any combination thereof. In certain embodiments, the binding of the differentiation factor to one or more Notch receptors activates the Notch signaling pathway and induces T cell lineage differentiation.

In certain embodiments, the differentiation factor is a Delta-like 4 (DLL-4). DLL-4 is a protein that is a homolog of the Drosophila Delta protein. The Delta protein family includes Notch ligands that are characterized by a DSL domain, EGF repeats, and a transmembrane domain.

In certain embodiments, the differentiation factor polypeptides are isolated from endogenous sources or synthesized in vivo or in vitro. Endogenous differentiation factor polypeptides may be isolated from healthy human tissue. Synthetic differentiation factor polypeptides are synthesized in vivo following transfection or transformation of template DNA into a host organism or cell, e.g., a mammal or cultured human cell line. Alternatively, synthetic differentiation factor polypeptides are synthesized in vitro by cell free translation or other art-recognized methods Sambrook, J., Fritsch, E. F., and Maniatis, T., Molecular Cloning: A Laboratory Manual. Cold Spring Harbor Laboratory Press, NY, Vol. 1, 2, 3 (1989), herein incorporated by reference).

In certain embodiments, differentiation factor polypeptides may be recombinant. In some embodiments, the differentiation factor polypeptides are humanized derivatives of mammalian differentiation factor polypeptides. Exemplary mammalian species from which the differentiation factor polypeptides are derived include, but are not limited to, mouse, rat, hamster, guinea pig, ferret, cat, dog, monkey, or primate. In some embodiments, the differentiation factor is a recombinant human protein. In some embodiments, the differentiation factor is a recombinant murine (mouse) protein. In some embodiments, the differentiation factor is a humanized derivative of a recombinant mouse protein.

In certain embodiments, the differentiation factor polypeptides may be modified to achieve a desired activity, for example, to increase protein stability in vivo. In certain embodiments, the differentiation factor polypeptides may be engineered to be more or less immunogenic.

In certain embodiments, the differentiation factor (e.g., DLL-4) may be covalently linked to the scaffold of the present invention. For example, rather than being released from a scaffold material, a differentiation factor may be covalently bound to polymer backbone and retained within the composition that forms following implantation of the composition in the subject. By covalently binding or coupling a differentiation factor to the scaffold material, such differentiation factor will be retained within the scaffold that forms following administration of the composition to a subject, and thus will be available to promote the differentiation of stem cells or progenitor cells, as contemplated herein. In certain embodiments, the differentiation factors are conjugated to the scaffold material utilizing N-hydroxysuccinimide (NHS) and 1-ethyl-3-(3-dimethylaminopropyl) carbodiimide hydrochloride (EDC) chemistry. Any methods of covalently binding or coupling differentiation factors known in the art may be used and are not limited. See “Bioconjugate Techniques Bioconjugate Techniques (Third Addition)”, Greg T. Hermanson, Academic, Greg T. Hermanson, Academic Press, 2013 Press, 2013. In some embodiments, the differentiation factor may be covalently linked to the scaffold utilizing click chemistry. The methods of covalently binding or coupling differentiation factors include, but are not limited to, avidin-biotin reaction, azide and dibenzocycloocytne chemistry, tetrazine and transcyclooctene chemistry, tetrazine and norbornene chemistry, or di-sulfide bond.

In certain embodiments, the differentiation factors (e.g., DLL-4) of the present invention further comprise a tether (e.g., PEG, PEG₂ₖ) and a methacrylate group (MA). In certain embodiments, the differentiation factor is methacrylated DLL-4-PEG₂ₖ.

In certain embodiments, the covalent linking retains the differentiation factors within the scaffold to provide the differentiation signal to the recruited cells in the scaffold. For example, less than 1% of the total differentiation factor is detected outside of the scaffold. The bioactivity of the differentiation factor may be retained for an extended period of time, such as at least three months after incorporation to the scaffold. The bioactivity of the differentiation factors may be measured by any appropriate methods, such as a colorimetric assay for DLL-4.

In certain embodiments, the differentiation factors may be present at between about 0.01 nmol and about 1000 nmol, about 0.1 nmol and about 100 nmol, or about 1 nmol and about 10 nmol per scaffold.

In some embodiments, the differentiation factors may be present at between about 1 ng and about 1000 micrograms per scaffold. In some embodiments, the differentiation factors may be present at between about 1 ng and about 100 micrograms per scaffold. For example, the differentiation factor may be present at between about 10 ng and about 500 μg, between about 50 ng and about 250 μg, between about 100 ng and about 200 μg, between about 1 μg and about 100 μg, between about 1 μg and about 50 μg, between about 1 μg and about 25 μg, between about 1 μg and about 10 μg, between about 2 μg and about 10 μg, or about 6 μg.

In various embodiments, the amount of differentiation factor present in a scaffold may vary according to the size of the scaffold. For example, the differentiation factor may be present at about 0.03 ng/mm³ (the ratio of the amount of differentiation factor in weight to the volume of the scaffold) to about 350 μg/mm³, such as between about 0.1 ng/mm³ and about 300 μg/mm³, between about 1 ng/mm³ and about 250 μg/mm³, between about 10 ng/mm³ and about 200 μg/mm³, between about 0.1 μg/mm³ and about 100 μg/mm³, between about 0.1 μg/mm³ and 50 about μg/mm³, or between about 0.1 μg/mm³ and about 20 μg/mm³, between about 0.1 μg/mm³ and about 10 μg/mm³, between about 0.1 μg/mm³ and about 5 μg/mm³, between about 0.1 μg/mm³ and about 1 μg/mm³, between about 0.1 μg/mm³ and 0.5 μg/mm³, or about 0.2 μg/mm³.

In certain embodiments, the DLL-4 may be present at about 6 μg per scaffold.

### Homing Factors

In certain embodiments, the composition of the present invention may further comprise a homing factor. As used herein, the term “homing factor” refers to an agent that is capable of inducing directed movement of a cell, e.g., a stem cell or a progenitor cell. In certain embodiments, the homing factors of the present invention are signaling proteins that can induce directed chemotaxis in nearby responsive cells. In various embodiments, the homing factors are cytokines and/or chemokines.

In certain embodiments, the inclusion of such homing factors in the compositions of the present invention promotes the homing of cells (e.g., transplanted stem cells and/or progenitor cells) to the scaffold composition administered to a subject. In certain aspects, such homing factors promote the infiltration of the cells (e.g., transplanted stem cells or progenitor cells) to the scaffold composition administered to the subject. In some embodiments, the homing factors comprise stromal cell derived factor (SDF-1). In certain embodiments, the homing factors are encapsulated in the material. In certain embodiments, the homing factors are released from the material over an extended period of time (e.g., about 7-30 days or longer, about 17-18 days).

In certain embodiments, the homing factors retain their bioactivity over an extended period of time. The bioactivity of the growth factor may be measured by any appropriate means. In certain example, the homing factors retain their bioactivity for at least 10 days, 12 days, 14 days, 20 days, or 30 days after the incorporation of the homing factors into the scaffold.

In some embodiments, the homing factors may be present at between about 0.01 nmol and about 1000 nmol, about 0.1 nmol and about 100 nmol, or about 1 nmol and about 10 nmol per scaffold.

In some embodiments, the homing factors may be present at between about 1 ng and about 1000 micrograms per scaffold. In some embodiments, the homing factors may be present at least about 100 micrograms. For example, the homing factor may be present at between about 10 ng and about 500 μg, between about 50 ng and about 250 μg, between about 100 ng and about 200 μg, between about 1 μg and about 100 μg, between about 1 μg and about 50 μg, between about 1 μg and about 25 μg, between about 1 μg and about 10 μg, between about 2 μg and about 10 μg, or about 6 μg.

In various embodiments, the amount of differentiation factor present in a scaffold may vary according to the size of the scaffold. For example, the differentiation factor may be present at about 0.03 ng/mm³ (the ratio of the amount of differentiation factor in weight to the volume of the scaffold) to about 350 μg/mm³, such as between about 0.1 ng/mm³ and about 300 μg/mm³, between about 1 ng/mm³ and about 250 μg/mm³, between about 10 ng/mm³ and about 200 μg/mm³, between about 0.1 μg/mm³ and about 100 μg/mm³, between about 0.1 μg/mm³ and 50 about μg/mm³, or between about 0.1 μg/mm³ and about 20 μg/mm³, between about 0.1 μg/mm³ and about 10 μg/mm³, between about 0.1 μg/mm³ and about 5 μg/mm³, between about 0.1 μg/mm³ and about 1 μg/mm³, between about 0.1 μg/mm³ and 0.5 μg/mm³, or about 0.2 μg/mm³.

### Exemplary Scaffold Compositions

The present invention provides scaffold composition for modulating the immune system in a subject. The compositions of the present invention include a porous scaffold, a growth factor present at an amount effective for inducing formation of a tissue or an organ with the scaffold and recruiting a cell, such as an immune cell (e.g., a hematopoietic stem cell (HSC) and/or a hematopoietic progenitor cell (HPC)), into the scaffold, and a differentiation factor that induces the differentiation of the recruited cell, e.g., into a T cell progenitor cell.

In one aspect, the present invention provides a composition for modulating the immune system in a subject, including a porous scaffold; a growth factor present at between about 1 ng to about 1000 μg per scaffold, and in an amount effective for inducing formation of a tissue or an organ within the scaffold and recruiting a cell into the scaffold; and a differentiation factor that induces the differentiation of the recruited cell into a lymphocyte T cell progenitor cell. For example, the growth factors may be present at an amount between about 1 ng and about 500 μg, between about 1 ng and about 200 μg, between about 1 ng and about 100 μg, between about 1 ng and about 50 μg, or between about 1 ng and about 10 μg.

In another aspect, the present invention provides a composition for modulating the immune system in a subject, including a porous scaffold; a growth factor present at between about 1 ng to about 1000 ng per scaffold, and in an amount effective for inducing formation of a tissue or an organ within the scaffold and recruiting a cell into the scaffold; and a differentiation factor that induces the differentiation of the recruited cell into a lymphocyte T cell progenitor cell. In more particular embodiments, the growth factors are present at between about 5 ng to about 500 ng, between about 5 ng to about 250 ng, between about 5 ng to about 200 ng, or at about 200 ng.

In yet a further aspect, the present invention is directed to a composition for modulating the immune system in a subject, including a porous scaffold; a growth factor present at between about 0.03 ng/mm³ to about 350 ng/mm³ by volume of scaffold, and in an amount effective for inducing formation of a tissue or an organ within the scaffold and recruiting a cell into the scaffold; and a differentiation factor that induces the differentiation of the recruited cell into a T cell progenitor cell. In various embodiments, the growth factor may be present at about 0.1 ng/mm³ and about 300 ng/mm³, between about 0.5 ng/mm³ and about 250 ng/mm³, between about 1 ng/mm³ and about 200 ng/mm³, between about 2 ng/mm³ and about 150 ng/mm³, between about 3 ng/mm³ and about 100 ng/mm³, between about 4 ng/mm³ and about 50 ng/mm³, between about 5 ng/mm³ and 25 ng/mm³, between about 6 ng/mm³ and about 10 ng/mm³, or between about 6.5 ng/mm³ and about 7.0 ng/mm³.

The composition may be designed to release growth factors in a controlled manner. The reduced quantities of the growth factor and/or the controlled release offers advantages over the art, such as reduced toxicity associated with the use of high level of growth factors, e.g., BMP-2, and suboptimal release kinetics.

In various embodiments, the growth factors are a bone morphogenetic protein, such as BMP-2, BMP-4, BMP-7, BMP-12, BMP-14, or any combination thereof. In some embodiments, the growth factors are BMP-2. In certain embodiments, the growth factors are a TGF-β, such as TGF-β1, TGF-β2, TGF-β3, TGF-β4, or any combination thereof. In a particular embodiment, the growth factor includes TGF-β1.

The porous scaffolds of the compositions according to the present invention can be any biocompatible and biodegradable scaffolds. In certain embodiments, the porous scaffolds comprises a hydrogel or cryogel. In various embodiments, the hydrogel or cryogel comprises an alginate or an alginate derivative, a gelation or a gelatin derivative, or a hyaluronic acid or a hyaluronic acid derivative.

In certain embodiments, the differentiation factors of the present invention comprise a polypeptide that binds to a Notch receptor. In various embodiments, the differentiation factors are selected from the group consisting of a Delta-like 1 (DLL-1), a Delta-like 2 (DLL-2), a Delta-like 3 (DLL-3), a Delta-like 3 (DLL-3), a Delta-like 4 (DLL-4), a Jagged 1, a Jagged 2, and any combination thereof. In some embodiments, the differentiation factors comprise DLL-4. In certain embodiments, the differentiation factors are covalently linked to the porous scaffold.

In a particular embodiments, the compositions of the present invention comprise an injectable cryogel, which comprises an alginate or an alginate derivative, such as methacrylated alginate, or a hyaluronic acid or a hyaluronic acid derivative; a growth factor that is a bone morphogenetic protein, such as BMP-2; and a differentiation factor that is a Delta-like family protein, such as DLL-4. The growth factor may be present at about 200 ng per scaffold or between about 6.5 ng/mm³ and 7.0 ng/mm³.

### III. Methods of Modifying Immune Cells and In Vivo Gene Therapy

The present invention features methods of modifying the immune cells of a subject in vivo or in situ. In certain embodiments of the present inventions, the methods of modifying the immune cells of the subject comprise (a) administering to the subject one or more compositions of the present invention, wherein the scaffold composition: (i) is a porous scaffold material; and (b) administering an active agent into the scaffold composition in situ. The scaffold composition provided herein can be used to recruit immune cells (e.g., hematopoietic stem cells (HSCs) and/or hematopoietic progenitor cells (HPCs)) to form a bone marrow niche (e.g., at the site of the administered scaffold) in a subject. Notably, the scaffold compositions provided herein can mimic key features of the bone marrow microenvironment, also called the bone marrow niche, and can be used to promote the regeneration of immune cells (e.g., T cells) in a subject after hematopoietic stem cell transplantation (HSCT). In certain embodiments, the scaffold compositions provided herein can be used to promote the regeneration of immune cells (e.g., T cells) in a subject prior to hematopoietic stem cell transplantation (HSCT). In certain embodiments, the scaffold compositions provided herein can be used to promote the regeneration of immune cells (e.g., T cells) in a subject without, or in the absence of, hematopoietic stem cell transplantation (HSCT).

Without wishing to be bound by theory, subcutaneous administration of the scaffold compositions provided herein to a subject can result in the formation of an interface with the subject's vasculature to form a subject-device interface, and to present lineage-instructive cues to recruited hematopoietic progenitor cells (HPCs) in vivo. In certain embodiments, the incorporation of a bioactive Notch ligand, such as DLL-4, on the polymer scaffold can promote the early enhancement in the generation of T-cell progenitors in the administered scaffold composition and lead to a significant increase in the number of thymic progenitors in the subject, for example, relative to subjects receiving lineage-depleted bone marrow grafts. The functional activities of the scaffold composition provided herein are consistent with that of a pre-thymic niche, promoting an increase in the number of T-cell progenitors that then traffic to the thymus to enhance immune reconstitution.

Advantageously, the scaffold compositions provided herein can enable localized transduction of immune cells (e.g., hematopoietic stem cells (HSCs) and/or hematopoietic progenitor cells (HPCs)) in vivo. Such scaffold compositions can be used, even in the absence of hematopoietic stem cell transplantation (HSCT), to recruit immune cells (e.g., hematopoietic stem cells (HSCs) and/or hematopoietic progenitor cells (HPCs)) to the scaffold material in a matter of days, e.g., e.g., 1, 2, 3, 4, 5, 6, or 7 days, or weeks, e.g., 1, 2, 3, 4, or 5 weeks, from administration and to rebuild a functional bone marrow niche (e.g., at the site of the administered scaffold) with physical properties and cellular composition similar to the endogenous bone marrow (FIG. 1A). In certain embodiments, the recruited immune cells (e.g., hematopoietic stem cells (HSCs) and/or hematopoietic progenitor cells (HPCs)) comprise transplanted hematopoietic stem cells (HSCs) and/or transplanted hematopoietic progenitor cells (HPCs). In certain embodiments, the recruited immune cells (e.g., hematopoietic stem cells (HSCs) and/or hematopoietic progenitor cells (HPCs)) comprise endogenous hematopoietic stem cells (HSCs) and/or endogenous hematopoietic progenitor cells (HPCs).

Injection of an active agent, such as a viral or non-viral expression vector, into the scaffold composition, can enable highly efficient transduction of immune cells (e.g., hematopoietic stem cells (HSCs) and/or hematopoietic progenitor cells (HPCs)) recruited to and localized in the scaffold (FIGS. 1B-1C). The methods provided herein may result in the local transduction of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 99% or more of hematopoietic progenitor cells (HPCs) and/or hematopoietic stem cells (HSCs) recruited to the scaffold composition in situ. In some embodiments, the method provided herein can result in the local transduction of between about 1% and about 50% of hematopoietic progenitor cells (HPCs) and/or hematopoietic stem cells (HSCs) recruited to the scaffold composition in situ. In some embodiments, the methods provided herein may result in the local transduction of between about 1% and about 25% of hematopoietic progenitor cells (HPCs) and/or hematopoietic stem cells (HSCs) recruited to the scaffold composition in situ. In another embodiment, the method results in the local transduction of between about 25% and about 50% of hematopoietic progenitor cells (HPCs) and/or hematopoietic stem cells (HSCs) recruited to the scaffold composition in situ. In some embodiments, the hematopoietic stem cells (HSCs) recruited to and localized in the scaffold comprise myeloid and/or lymphoid hematopoietic cells.

In certain embodiments, as compared to direct intra-femoral delivery in the bone marrow, injection of the same dose of a vector in the administered scaffold composition can result in at least about a 1-fold, about a 2-fold, about a 3-fold, about a 4-fold, about a 5-fold, about a 6-fold, about a 7-fold, about a 8-fold, about a 9-fold, about a 10-fold or higher transduction in the hematopoietic progenitor cells (HPCs) and/or hematopoietic stem cells (HSCs) compartment (FIG. 1D).

Direct injection of viral and non-viral agents into the scaffold compositions has several advantages over systemic intravenous administration, including, for example a reduction of the vector dose needed for effective hematopoietic progenitor cells (HPCs) and/or hematopoietic stem cells (HSCs) transduction; and reduction of innate and adaptive immune responses. For example, systemic intravenous administration of viral particles can be sensed by several immune mechanisms and is associated with very pronounced inflammatory storms that often require pre-treatment with immunosuppressive drugs.

Additionally, the scaffold compositions described herein provide a high portability of treatment. A practical aspect of the scaffold composition treatment is that it can be administered without hospitalization, can be cryopreserved, and requires no ex vivo culturing. Moreover, the scaffold compositions are not patient specific, and the same scaffold compositions can be used for all subjects. Therefore, in certain embodiments, the scaffold compositions provided herein can serve as an off the shelf reagent administered by simple subcutaneous injection. In certain embodiments, the scaffold compositions can be injected into the sub-clavicular space of a subject, similar to where other devices such as vascular ports or pacemakers can be located without significant morbidity or discomfort for subjects. In some embodiments, a plurality of scaffold compositions may be administered, e.g., simultaneously and/or sequentially, to a subject. In some embodiments, two or more (e.g., 3, 4, 5, 6, 7, 8, 9, or 10 or more) scaffold compositions may be administered, e.g., simultaneously and/or sequentially, to a subject. Each scaffold composition may also be removed and/or replaced, independently, at need with a simple skin incision and the use of a forceps.

In terms of volume, in some embodiments, the size of the scaffold composition can have a size of between about 1 in³ and about 10 in³. For example, the scaffold composition can have a size of about 1, 2, 3, 4, 5, 6, 7, 8, 9, or 10 or more in³.

In various embodiments of the methods delineated herein, the active agent comprises an amino acid, a peptide, a protein, a nucleic acid, an oligonucleotide, a small molecule, or a combination thereof. In some embodiments, the active agent comprises a nucleic acid. In some embodiments, the active agent comprises a vector. In some embodiments, the active agent comprises an expression vector. In some embodiments, the active agent comprises a viral vector. In some embodiments, the active agent comprises a viral vector selected from the group consisting of a retrovirus vector, a herpes simplex vector, a lentivirus vector, an adenovirus vector, and an adeno-associated virus vector. In some embodiments, the active agent comprises a lentivirus vector. In some embodiments, the active agent comprises a nucleic acid sequence encoding a gene product.

In various embodiments of the methods delineated herein, the method further comprises administering an additional dose of the active agent into the scaffold.

In some embodiments, the method further comprises administering an additional active agent into the scaffold.

In some embodiments, the method results in modification of an immune cell (e.g., a hematopoietic stem cell (HSC) and/or a hematopoietic progenitor cell (HPC)) recruited to the scaffold composition in situ to replace a polynucleotide or gene product, or to add or knockdown a gene product.

In various embodiments of the methods delineated herein, the scaffold material is a hydrogel. In some embodiments, the scaffold material is a cryogel. In some embodiments, the scaffold material comprises a polymer or co-polymer selected from the group consisting of polylactic acid, polyglycolic acid, PLGA, alginate or an alginate derivative, gelatin, collagen, fibrin, agarose, hyaluronic acid, poly(lysine), polyhydroxybutyrate, poly-epsilon-caprolactone, polyphosphazines, poly(vinyl alcohol), poly(alkylene oxide), poly(ethylene oxide), poly(allylamine), poly(acrylate), poly(4-aminomethylstyrene), pluronic polyol, polyoxamer, poly(uronic acid), poly(anhydride), poly(vinylpyrrolidone), and any combination thereof. In some embodiments, the scaffold material comprises a polymer or co-polymer selected from the group consisting of alginate, alginate derivative, and any combination thereof. In some embodiments, the scaffold material comprises alginate. In some embodiments, the scaffold material comprises methacrylated alginate (MA-alginate). In some embodiments, the scaffold material comprises anionic alginate. In some embodiments, the scaffold material comprises a polymer or co-polymer selected from the group consisting of hyaluronic acid, hyaluronic acid derivative, and any combination thereof. In some embodiments, the scaffold material comprises a hyaluronic acid or a hyaluronic acid-derivative. In some embodiments, the scaffold material comprises a click-hydrogel or a click cryogel. In some embodiments, the scaffold material comprises a click-alginate, a click gelatin, or a click hyaluronic acid.

In various embodiments of the methods delineated herein, the scaffold material comprises pores having a diameter between about 1 nm and about 100 μm. In some embodiments, the scaffold material comprises a macropore. In some embodiments, the macropore has a diameter between about 20 μm and about 80 μm. In some embodiments, the macropore has a diameter between about 50 μm and about 80 μm. In some embodiments, the scaffold material comprises macropores of different sizes.

In various embodiments of the methods delineated herein, the scaffold composition is implantable and/or injectable.

In various embodiments of the methods delineated herein, the growth factor comprises a bone morphogenetic protein (BMP). In some embodiments, the growth factor is selected from the group consisting of a BMP-2, a BMP-4, a BMP-6, a BMP-7, a BMP-12, a BMP-14, and combinations thereof. In some embodiments, the growth factor comprises a BMP-2. In some embodiments, the growth factor is encapsulated in the scaffold material. In some embodiments, the growth factor is released from the scaffold material over about 7-30 days.

In various embodiments of the methods delineated herein, the differentiation factor binds to a Notch receptor. In some embodiments, the Notch receptor is selected from the group consisting of a Notch-1 receptor, a Notch-2 receptor, a Notch-3 receptor, a Notch-4 receptor, and any combination thereof. In some embodiments, the differentiation factor is selected from the group consisting of a Delta-like 1 (DLL-1), a Delta-like 2 (DLL-2), a Delta-like 3 (DLL-3), a Delta-like 4 (DLL-4), a Jagged 1, a Jagged 2, and any combination thereof. In some embodiments, the differentiation factor is bound, directly or indirectly, to the scaffold material. In some embodiments, the differentiation factor is covalently bound to the scaffold material or covalently bound to a tether that is covalently bound to the scaffold material.

In various embodiments of the methods delineated herein, the scaffold composition further comprises a cytokine. In some embodiments, the cytokine comprises interleukin-7 (IL-7). In some embodiments, the cytokine is encapsulated in the scaffold material. In some embodiments, the cytokine is released from the scaffold material over about 7 to about 30 days.

In various embodiments of the methods delineated herein, the scaffold composition further comprises a homing factor. In some embodiments, the homing factor comprises a stem cell differentiation factor (SDF-1). In some embodiments, the homing factor is encapsulated in the scaffold material. In some embodiments, the homing factor is released from the scaffold material over about 7 to about 30 days.

In various embodiments of the methods delineated herein, (i) the growth factor promotes formation of tissue on or around the administered scaffold material to form a bone marrow niche; (ii) the differentiation factor promotes the differentiation of a stem cell to a lymphoid lineage cell; and/or (iii) the homing factor promotes the infiltration of a stem cell and/or a progenitor cell to the bone marrow niche.

In some embodiments, the stem cell is a transplanted stem cell. In some embodiments, the stem cell is a hematopoietic stem cell.

In various embodiments of the methods delineated herein, the method results in (i) transduction of an immune cell localized in the scaffold material in vivo, optionally wherein the immune cell comprises a hematopoietic stem cell (HSC), optionally wherein the HSC comprises a myeloid and/or a lymphoid hematopoietic cell; (ii) greater transduction of an immune cell, optionally a primitive Lin− Kit+ Sca+ hematopoietic cell (HSC), localized in the scaffold material in vivo as compared to interfemoral injection of the same dose of active agent, optionally, by at least about 5%, or at least about 10%, or at least about 20%, or at least about 30%, or at least about 40%, or at least about 50%, or at least about 60%, or at least about 70%, or at least about 80%, or at least about 90%, or at least about 95%, or at least about 99%, or up to and including a 100% increase, or any increase between about 5 and about 100%; (iii) recruitment of endogenous and/or transplanted immune cells, optionally hematopoietic stem cells (HSC), to the scaffold material within about 1-3 weeks after administration; and/or (iv) an increase in the number of immune cells, optionally T-cell competent progenitor cells, that traffic to the thymus to enhance immune reconstitution.

In various embodiments of the methods delineated herein, the subject is a human. In certain embodiments, the methods of the present invention modulate the immune response of a human over 30 years of age. For example, the human may be over 40, over 50, over 60, over 70, over 80 years of age.

In some embodiments, the scaffold composition is administered to the subject via injection, optionally, intravenously, intramuscularly, or subcutaneously. In some embodiments, the scaffold composition is administered to the subclavicular fossa of the subject.

In certain embodiments, at least two compositions are administered to the subject. The compositions can be of similar size.

In various embodiments of the methods delineated herein, the subject has or is receiving a hematopoietic stem cell transplantation (HSCT). In some embodiments, the administration of the composition is prior to, concurrently with, or subsequent to the HSCT. Hematopoietic stem cell transplantation (HSCT) is a curative treatment for multiple disorders, but allogeneic HSCT is limited by deficiency and dysregulation of T-cells. In a subject that receives allogeneic HSCT, CD4+ T-cell recovery is usually delayed, leading to an inversion of the normal CD4/CD8 ratio, which is about 0.9 to about 2.5 in periphery blood. The ratio may be different in other tissue or organ. In certain embodiments, the methods of the present invention stabilize the CD4+: CD8+ ratio to a normal range, while CD4+ T-cell compartment in a subject receiving HSCT only has not fully reconstituted. In certain embodiments, a balanced T cell reconstitution is characterized by homeostatic CD4+: CD8+ ratio in a normal range in 30 days or less after the transplantation of the hematopoietic stem cells and the administration of the composition of the present invention. The term “balanced reconstitution of T cells,” as used herein, refers to the reconstitution of T cells that is characterized by CD4+: CD8+ ratio in a normal range within a certain period of time, such as 30 days. For example, the reconstitution of CD4+ cells is usually delayed in a HSCT recipient. The methods of the present invention may accelerate the reconstitution of CD4+ T cells and lead to a balanced reconstitution of T cells.

In certain embodiments according to the present invention, a subject, such as a human, receives between about 1×105 and about 50×106 hematopoietic stem cells or progenitor cells per kilogram of the subject's weight in a hematopoietic stem cell transplantation. In certain embodiments, the subject receives about 1×105 hematopoietic stem cells per kilogram of the subject's weight.

The methods of the present invention result in similar or better curative and/or therapeutic effects when compared to a subject that receives hematopoietic stem cell or T-cell progenitor infusion alone (i.e., without receiving the treatment of the compositions of the present invention). For example, treatment with the compositions of the present invention may result in a higher number of T-cell progenitors and functional T-cells in the thymus and the periphery, for example, even when used with a lower dose relative to T-cell progenitor infusion alone. In some embodiments, similar or better curative and/or therapeutic effects can be achieved when less than ten percent (10%) of hematopoietic stem cells or progenitor cells used in a HSCT alone are administered to a subject in combination with the compositions of the present invention.

In certain embodiments, the methods further comprise administering to the subject a hematopoietic stem cell or a hematopoietic progenitor cell.

In certain embodiments, the cells are stem cells or progenitor cells. As used herein, the term “stem cell” refers to a biological cell that can differentiate into other types of cells and can divide to produce more of the same type of stem cells. Stem cells include embryonic stem cells, which are isolated from the inner cell mass of blastocysts, and adult stem cells, which are found in various tissues. In adult organisms, stem cells and progenitor cells act as a repair system for the body, replenishing adult tissues. In certain embodiments, the stem cells are embryonic stem cells, fetal stem cells, amniotic stem cells, umbilical cord stem cells, adult stem cells, or induced pluripotent stem cells. In certain embodiments, the stem cells are hematopoietic stem cells. Hematopoietic stem cells are the stem cells that give rise to other blood cells, including both myeloid and lymphoid lineage of blood cells.

As used herein, the term “progenitor cell” refers to a biological cell that can differentiate into a specific type of cell. Progenitor cells are generally more differentiated than stem cells. Typically, progenitor cells can only divide a limited number of times.

In certain embodiments, the progenitor cells are blast cells, such as thymocytes, lymphoblasts, myeloid, or bone marrow precursor cells. In certain embodiments, the progenitor cells are cells that are capable of differentiating into T cell progenitor cells. In certain embodiments, the lymphocytes include T cells, such as naïve T cells.

In certain embodiments, the recruited cells are hematopoietic bone marrow cells, or mobilized peripheral blood cells.

In certain embodiments, the cells may be recombinant cells. The term “recombinant cell,” as used herein, refers to a cell into which a genetic modification has been introduced. The genetic modification may be at chromosomal level or extra-chromosomal. “Genetic modification at chromosomal level” refers to the genetic modification in the genome of the cell, e.g., insertion, deletion, and/or substitution on the chromosome of the cell. Extra-chromosomal genetic modification refers to the genetic modification not located in the genome of the cell. For example, a plasmid containing a protein encoding gene may be introduced to the cell. The plasmid may replicate and transmit from parental cells to offspring cells.

In various embodiments, the genetic modification introduces a gene into the cell. The introduced gene may compensate for the function of a defective gene of the cell. For example, the cell may contain a mutant defective gene. The genetic modification may introduce a wild type functional gene into the cell to restore the function of the gene. In some embodiment, the genetic modification may increase or decrease the expression of certain gene. For example, the genetic modification may introduce a small interfering RNA (siRNA) specific to a gene to inhibit the expression of the gene.

The methods to genetically modify a cell are commonly known in the art such as the methods described in Sambrook, J., Fritsch, E. F., and Maniatis, T., Molecular Cloning: A Laboratory Manual. Cold Spring Harbor Laboratory Press, NY, Vol. 1, 2, 3 (1989), herein incorporated by reference.

In certain embodiments, the genetic modification may be introduced through gene editing, also known as genome editing. Gene editing is a group of technologies that give skilled artisans the ability to change an organism's DNA. These technologies allow genetic material to be added, removed, or altered at particular locations in the genome. Gene editing technologies include, but are not limited to, meganucleases system, Zinc finger nucleases (ZFN) system, transcription activator-like effector nucleases (TALENs) system, and CRISPR-Cas system. CRISPR-Cas systems, which is short for clustered regularly interspaced short palindromic repeats and CRISPR-associated protein systems, in particular CRISP-Cas9, is faster, cheaper, more accurate, and more efficient than other existing genome editing methods.

In certain embodiments, the present invention features methods that modulate the immune system of a subject after the subject receives a transplantation. For example, the subject may receive a hematopoietic stem cell transplantation. In certain embodiments, the compositions of the present invention are administered to the subject concurrently with, or after, the hematopoietic stem cell transplantation.

In some embodiments, one or more compositions of the present invention (e.g., bone marrow cryogels) may be administered in conjunction with stem cell mobilization techniques. Stem cell mobilization is a process by which certain cell mobilization agents are used to cause the movement of stem cells from the bone marrow into the blood, such as described in Hopman and DiPersio, Advances in Stem Cell Mobilization, Blood Rev., 2014, 28 (1): 31-40, the content of which is incorporated herein by reference. Such techniques may also be used for mobilization of progenitor cells.

Accordingly, in certain embodiments, a subject is administered a stem and/or progenitor cell mobilization agent in an amount effective to induce the movement of stem/progenitor cells from bone marrow into the blood. Released stem and/or progenitor cells are subsequently recruited to the composition of the present invention (e.g., a bone marrow cryogel) to differentiate into T cell progenitor cells. The stem and/or progenitor cell mobilization agent may be administered prior to, concurrently with, or following the administration of the composition (e.g., a bone marrow cryogel).

In various embodiments, the composition of the present invention (e.g., a bone marrow cryogel) may be administered to a subject in conjunction with a stem and/or progenitor cell mobilization agent. In particular embodiments, the subject is a human with advanced age, for example, the human may be over 30, 40, 50, 60, 70, or 80 years old. The stem and/or progenitor cell mobilization agent may mobilize the subject's own stem and/or progenitor cells out of the bone marrow so these cells can home to the composition of the present invention, thereby enhancing generation of T cells in the subject without involving other conditioning or stem cell transplant.

In certain embodiments, the composition of the present invention (e.g., a bone marrow cryogel) may be administered to a subject in conjunction with stem and/or progenitor cell mobilization techniques and stem cell transplantation. The transplantation may be autologous, allogeneic, or xenogeneic.

In some embodiments, a therapeutically-effective amount of one, or more cell mobilization agents that can stimulate mobilization into the peripheral bloodstream, production and/or improve function of one or more cell types is administered. The agent(s) could be given through any desired route of administration, including orally, rectally, intravenously, intramuscularly, subcutaneously, or an aerosol. Some non-limiting embodiments of an agent that can stimulate mobilization into the peripheral bloodstream, production of and/or improve function of a cell type include IL-1, IL-2, IL-3, IL-6, GM-CSF, G-CSF, plerixafor, PDGF, TGF-beta, NGF, IGFs, growth hormone, erythropoietin, thrombopoietin, and the like. In addition to naturally occurring growth factors, growth factor analogs and growth factor derivatives such as fusion proteins can be used as well. In some embodiments, the method involves administration of a therapeutically-effective amount of G-CSF and a therapeutically-effective amount of electromagnetic radiation. In some embodiments, the method comprises administering a combination of a therapeutically-effective amount of plerixafor and a therapeutically-effective amount of electromagnetic radiation. In some embodiments, a therapeutically-effective amount of electromagnetic radiation is combined with another agent that, in some embodiments, could be a hematopoietic stem cell mobilizer. In some embodiments, a therapeutically-effective amount of electromagnetic radiation is combined with combinations of two or more of G-CSF, GM-CSF, plerixafor, IL-1, IL-2, IL-3, IL-6, PDGF, TGF-beta, NGF, IGFs, growth hormone, erythropoietin, thrombopoietin or another agent.

### IV. Kits

Any of the compositions described herein may be included in a kit. In a non-limiting example, the kit includes a composition comprising a porous scaffold, a growth factor, and rand a differentiation. In some embodiments, the kit includes an active agent, such as a viral or non-viral expression vector.

In some embodiments, the kit includes the composition described elsewhere herein.

In a particular embodiment, the kit comprises a syringe or alternative injection device for administering the composition. In a specific embodiment, the prefilled syringe or injection device is prefilled with the composition.

The kit may further include reagents or instructions for administering the composition of the present invention to a subject. It may also include one or more reagents.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. The kits may also comprise a second container means for containing a sterile, pharmaceutically acceptable buffer and/or other diluent. However, various combinations of components may be comprised in a vial. The kits of the present invention also will typically include a means for containing the compositions of the invention, e.g., the compositions for modulating immune system, and any other reagent containers in close confinement for commercial sale.

When the components of the kit are provided in one and/or more liquid solutions, the liquid solution is an aqueous solution, with a sterile aqueous solution being particularly preferred. However, the components of the kit may be provided as dried powder(s). When reagents and/or components are provided as a dry powder, the powder can be reconstituted by the addition of a suitable solvent. It is envisioned that the solvent may also be provided in another container means.

The present invention is further illustrated by the following examples, which should not be construed as limiting. All cited sources, for example, references, publications, databases, database entries, and art cited herein, are incorporated into this application by reference, even if not expressly stated in the citation. In case of conflicting statements of a cited source and the instant application, the statement in the instant application shall control.

Section and table headings are not intended to be limiting.

### EXAMPLES

### Example 1: Scaffold Compositions Enable Localized Transduction of Hematopoietic Stem Cells (HSCs) In Vivo

The scaffold composition, described herein, was used to enable localized transduction of hematopoietic stem cells (HSCs) in vivo. The data showed that even in the absence of transplantation, endogenous hematopoietic stem cells (HSCs) are recruited to the administered scaffold composition within 3 weeks from administration, rebuilding a functional bone marrow niche with physical properties and cellular composition similar to the endogenous bone marrow (FIG. 1A). The scaffold composition used in this experiment was a cell-free biomaterial-based bone marrow cryogels (BMC), fabricated from click-alginate and included BMP-2 to induce bone formation as well as conjugated DLL-4 protein.

Intra scaffold injection of ˜10\hat{ }8 TU of highly concentrated GFP-expressing lentiviral vector (LV) enabled highly efficient transduction of myeloid and lymphoid hematopoietic cells localized in the scaffold composition, with a mean of ˜5% transduction in the HSC-enriched fractions (FIGS. 1B-1C). As compared to direct intra-femoral delivery in the bone marrow, injection of the same dose of lentiviral vector in the scaffold composition resulted in 10-fold higher transduction in the HSC compartment (FIG. 1D).

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the present invention described herein. Such equivalents are intended to be encompassed by the following claims.

1. A method of delivering an active agent to a subject in need thereof, comprising:
a. administering to a subject a scaffold composition, wherein the scaffold composition is a porous scaffold material; and
b. administering an active agent into the scaffold composition in situ,
thereby delivering the active agent to the subject, optionally to an immune cell in the subject.

2. A method of locally transducing an immune cell in a subject in need thereof, comprising:
a. administering to a subject a scaffold composition, thereby recruiting hematopoietic stem cells (HSCs) and/or a hematopoietic progenitor cells (HPCs) to form a bone marrow niche, wherein the scaffold composition is a porous scaffold material; and
b. administering an active agent into the scaffold,
thereby locally transducing the immune cell in the subject.

3. A method of promoting the regeneration of immune cells in a subject in need thereof, comprising:
a. administering to a subject a scaffold composition, wherein the scaffold composition is a porous scaffold material; and
b. administering an active agent into the scaffold,
thereby promoting the regeneration of immune cells.

4. The method of claim 1,
(i) wherein the scaffold composition recruits immune cells to form a bone marrow niche in situ;
(ii) wherein the method results in the local transduction of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 99% or more of hematopoietic progenitor cells (HPCs) and/or hematopoietic stem cells (HSCs) recruited to the scaffold composition in situ; and/or
(iii) wherein the method results in the local transduction of between about 1% and about 50% of hematopoietic progenitor cells (HPCs) and/or hematopoietic stem cells (HSCs) recruited to the scaffold composition in situ.

5-6. (canceled)

7. The method of claim 1,
(i) wherein the active agent comprises an amino acid, a peptide, a protein, a nucleic acid, an oligonucleotide, a vector, a small molecule, or a combination thereof;
(ii) wherein the active agent comprises a composition for modifying cells, optionally wherein the composition for modifying cells comprises a system for generating site-specific gene modifications;
(iii) wherein the active agent comprises a CRISPR/Cas9 system, an RNAi, a transcription activator-like effector nuclease (TALEN), a transcription activator-like effectors (TALEs), a meganuclease, a zinc finger nucleases (ZFN), or a combination thereof;
(iv) wherein the active agent comprises a gene therapy and/or a chimeric antigen receptor (CAR) T-cell therapy;
(v) wherein the active agent comprises an expression vector and/or a viral vector;
(vi) wherein the active agent comprises a viral vector selected from the group consisting of a retrovirus vector, a herpes simplex vector, a lentivirus vector, an adenovirus vector, and an adeno-associated virus vector;
(vii) wherein the active agent comprises a lentivirus vector; or
(viii) wherein the active agent comprises a nucleic acid sequence encoding a gene product.

8-14. (canceled)

15. The method of claim 1, wherein the method results in modification of immune cells recruited to the scaffold composition in situ to replace a polynucleotide or gene product, or to add or knockdown a gene product.

16. The method of claim 1,
(i) wherein the scaffold material is a hydrogel;
(ii) wherein the scaffold material is a cryogel;
(iii) wherein the scaffold material comprises a polymer or co-polymer selected from the group consisting of polylactic acid, polyglycolic acid, PLGA, alginate or an alginate derivative, gelatin, collagen, fibrin, agarose, hyaluronic acid, poly(lysine), polyhydroxybutyrate, poly-epsilon-caprolactone, polyphosphazines, poly(vinyl alcohol), poly(alkylene oxide), poly(ethylene oxide), poly(allylamine), poly(acrylate), poly(4-aminomethylstyrene), pluronic polyol, polyoxamer, poly(uronic acid), poly(anhydride), poly(vinylpyrrolidone), and any combination thereof;
(iv) wherein the scaffold material comprises a polymer or co-polymer selected from the group consisting of alginate, alginate derivative, and any combination thereof;
(v) wherein the scaffold material comprises alginate;
(vi) wherein the scaffold material comprises methacrylated alginate (MA-alginate);
(vii) wherein the scaffold material comprises anionic alginate;
(viii) wherein the scaffold material comprises a polymer or co-polymer selected from the group consisting of hyaluronic acid, hyaluronic acid derivative, and any combination thereof;
(ix) wherein the scaffold material comprises a hyaluronic acid or a hyaluronic acid-derivative;
(x) wherein the scaffold material comprises a click-hydrogel or a click cryogel; or
(xi) wherein the scaffold material comprises a click-alginate, a click gelatin, or a click hyaluronic acid.

17-26. (canceled)

27. The method of claim 1,
(i) wherein the scaffold material comprises pores having a diameter between about 1 nm and about 100 μm;
(ii) wherein the scaffold material comprises a macropore, optionally, wherein the macropore has a diameter between about 20 μm and about 80 μm;
(iii) wherein the scaffold material comprises macropores of different sizes; or
(iv) wherein the scaffold composition is implantable and/or injectable.

28-31. (canceled)

32. The method of claim 1, wherein the scaffold composition further comprises a growth factor, optionally,
(i) wherein the growth factor comprises a bone morphogenetic protein (BMP) selected from the group consisting of a BMP-2, a BMP-4, a BMP-6, a BMP-7, a BMP-12, a BMP-14, and combinations thereof;
(ii) wherein the growth factor comprises a BMP-2;
(iii) wherein the growth factor is encapsulated in the scaffold material;
(iv) wherein the growth factor is released from the scaffold material over about 7-30 days; and/or
(v) the growth factor promotes formation of tissue on or around the administered scaffold material to form a bone marrow niche.

33-36. (canceled)

37. The method of claim 1, wherein the scaffold composition further comprises a differentiation factor, optionally,
(i) wherein the differentiation factor binds to a Notch receptor selected from the group consisting of a Notch-1 receptor, a Notch-2 receptor, a Notch-3 receptor, a Notch-4 receptor, and any combination thereof;
(ii) wherein the differentiation factor is selected from the group consisting of a Delta-like 1 (DLL-1), a Delta-like 2 (DLL-2), a Delta-like 3 (DLL-3), a Delta-like 4 (DLL-4), a Jagged 1, a Jagged 2, and any combination thereof;
(iii) wherein the differentiation factor is bound, directly or indirectly, to the scaffold material;
(iv) wherein the differentiation factor is covalently bound to the scaffold material or covalently bound to a tether that is covalently bound to the scaffold material; and/or
(v) the differentiation factor promotes the differentiation of a stem cell to a lymphoid lineage cell.

38-41. (canceled)

42. The method of claim 1, wherein the scaffold composition further comprises a cytokine, optionally,
(i) wherein the cytokine comprises interleukin-7 (IL-7);
(ii) wherein the cytokine is encapsulated in the scaffold material; and/or
(iii) wherein the cytokine is released from the scaffold material over about 7-30 days.

43-45. (canceled)

46. The method of claim 1, wherein the scaffold composition further comprises a homing factor, optionally,
(i) wherein the homing factor comprises a stem cell differentiation factor (SDF-1);
(ii) wherein the homing factor is encapsulated in the scaffold material;
(iii) wherein the homing factor is released from the scaffold material over about 7-30 days; and/or
(iv) the homing factor promotes the infiltration of a stem cell and/or a progenitor cell to the bone marrow niche.

47-50. (canceled)

51. The method of claim 37, wherein the stem cell is a transplanted stem cell or a hematopoietic stem cell.

52. (canceled)

53. The method of claim 1, wherein the method results in
(i) transduction of an immune cell localized in the scaffold material in vivo, optionally wherein the immune cell comprises a hematopoietic stem cell (HSC), optionally wherein the HSC comprises a myeloid and/or a lymphoid hematopoietic cell;
(ii) greater transduction of an immune cell, optionally a primitive Lin− Kit+ Sca+ hematopoietic cell (HSC), localized in the scaffold material in vivo as compared to interfemoral injection of the same dose of active agent, optionally, by at least about 5%, or, at least about 10%, or at least about 20%, or at least about 30%, or at least about 40%, or at least about 50%, or at least about 60%, or at least about 70%, or at least about 80%, or at least about 90%, or at least about 95%, or at least about 99%, or up to and including a 100% increase, or any increase between about 5 and about 100%;
(iii) recruitment of endogenous and/or transplanted immune cells, optionally hematopoietic stem cells (HSC), to the scaffold material within about 1-3 weeks after administration; and/or
(iv) an increase in the number of immune cells, optionally T-cell competent progenitor cells, that traffic to the thymus to enhance immune reconstitution.

54. (canceled)

55. The method of claim 1, wherein the composition is administered to the subject via injection, optionally, intravenously, intramuscularly, or subcutaneously; and/or wherein the scaffold is administered to the subclavicular fossa of the subject.

56. (canceled)

57. The method of claim 1, wherein the subject has or is receiving a hematopoietic stem cell transplantation (HSCT).

58. The method of claim 57, wherein the administration of the composition is prior to, concurrently with, or subsequent to the HSCT.

59. The method of claim 1, further comprising administering an additional dose of the active agent into the scaffold and/or administering an additional active agent into the scaffold.

60. (canceled)

61. The method of claim 1, wherein the scaffold is cell free prior to administration to the subject.

62. The method of claim 1,
(a) wherein the immune cell comprises
(i) a stem cell and/or progenitor cell, optionally wherein the stem cell and/or the progenitor cell comprises a hematopoietic stem cell (HSC) and/or a hematopoietic progenitor cell (HPC);
(ii) a lymphocyte, optionally wherein the lymphocyte comprises a T cell, a B cell, and/or a natural killer (NK) cell;
(iii) a myeloid-derived cell, optionally wherein the myeloid-derived cell comprises a neutrophil, an eosinophil, a basophil, a monocyte, a macrophage, and/or a dendritic cell;
(iv) a modified immune cell; and/or
(v) any combination thereof; and/or
(b) wherein the subject has not received a hematopoietic stem cell transplantation (HSCT).

63-65. (canceled)
